(19) United States
(12) Patent Application Publication
(10) Pub. No.: US 2025/0366830 A1
(43) Pub. Date: Dec. 4, 2025

(54) ARTIFICIAL INTELLIGENCE MODELS FOR INTERPRETATION OF POINT-OF-CARE ULTRASOUND EXAMINATIONS

(71) Applicant: The Government of The United States, as represented by The Director of The Defense Health Agency, Fort Detrick, MD (US)

(72) Inventors: Sofia I. Hernández Torres, San Antonio, TX (US); Emilee C. Venn, AP APO, CA (US); Eric J. Snider, San Antonio, TX (US)

(73) Assignee: The Government of The United States, as represented by The Director of The Defense Health Agency, Frederick, MD (US)

(21) Appl. No.: 19/220,913

(22) Filed: May 28, 2025

Related U.S. Application Data
(60) Provisional application No. 63/652,891, filed on May 29, 2024.

(51) Int. Cl. A61B 8/00; G06T 3/40; G06T 7/00; G06V 10/72; G06V 10/82; G16H 50/20

(52) U.S. Cl. CPC A61B 8/5223; G06T 3/40; G06T 7/0012; G16H 50/20; A61B 2503/40; G06T 2207/10016; G06T 2207/10132; G06T 2207/20081; G06T 2207/20084; G06T 2207/30004; G06T 2207/30212; G06V 10/72; G06V 10/82; G06V 2201/03

(57) ABSTRACT

A system and a method for automatically predicting an internal trauma of a patient. Point of care ultrasound (POCUS) images of the patient are processed, with each POCUS image associated with a scan site of the patient, in accordance with selected scan sites of the patient and augmentation settings to generate processed POCUS images of the patient. The processed POCUS images associated with the one or more selected scan sites and augmentation settings are interpreted using one or more trained AI models to automatically generate a predicted internal trauma injury result at a selected scan site of the patient.

### PRIORITY CLAIM

This application claims the benefit of provisional application Ser. No. 63/652,891 filed May 29, 2024 and titled “Artificial Intelligence Models for Interpretation of Canine Point-of-Care Ultrasound Examinations” the entire contents of which are hereby incorporated by reference.

### STATEMENT OF GOVERNMENT INTEREST

The invention described herein may be manufactured, used and licensed by or for the United States Government.

### BACKGROUND

Working dogs, such as military working dogs (MWDs), are of critical importance in a number of working environments such as on the battlefield, working alongside soldiers in most functions. That can result in similar injury outcomes for MWDs as for soldier, and without the needed veterinarian assistance. Triaging an injured MWD can be challenging as veterinary expertise is not likely to be available in the far forward environment where injuries are likely to occur. This is particularly the case where point of care ultrasound (POCUS) is critical to making informed triage decisions in critical care medicine and prehospital care. As used herein, MWD is interchangeable with working dog, dog, canine, etc.

Ultrasound may be used in canines with suspected abdominal or thoracic injuries following trauma to identify free fluid which may require surgical intervention. Different standardized exams are used in veterinary medicine such as the abdominal Focused Assessment with Sonography for Trauma (AFAST®), thoracic FAST (TFAST®), or the Veterinary Bedside Lung Ultrasound Exam (Vet BLUE®) (Boysen and Lisciandro, 2013; Kate Boatright, 2020; Cole et al., 2021). These assessments are often performed together and referred to as GlobalFAST® which can be used for civilian trauma cases, but also for working dog casualties (Lisciandro and Lisciandro, 2021). Working dogs cover a wide range of occupations including military working dogs (MWDs) which go anywhere soldiers are deployed and aid with a wide range of tasks (Green, 2021). The ever increasing high risk mission that MWDs share with their handlers puts them at risk for similar injuries as their Service member counterparts. (Edwards et al., 2021; McGraw and Thomas, 2021). Unfortunately, in the early roles and stages of care, where MWD casualties are first managed, veterinary expertise may not be present to properly acquire ultrasound images and to interpret images making GlobalFAST® inaccessible for treatment of MWDs at these early stages of care (Lagutchik et al., 2018).

This is further complicated on the battlefield where medical evacuation will be limited and more medical care and triage will need to be provided in theater, at early stages of care. In fact, this is already being experienced with current conflicts in which limited medical evacuation opportunities arise due to challenged airspace, which is requiring far forward surgical teams to treat and manage a larger number of casualties for up to 72 hours in theater (Epstein et al., 2023). This is further complicated by precise long-range weaponry minimizing the relative safety of CASEVAC even at distances above 500 km away from enemy lines. In addition, more than 70% of Ukraine casualties stem from more advanced rocket or artillery injuries, which often result in complex polytrauma to multiple organ systems (Epstein et al., 2023). Thus, looking towards the battlefield, where access to evacuation is limited, it is even more imperative to have accurate triage procedures for prioritizing injured MWDs and other patients.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates prediction results by Scan Point/Site for MobileNetV2, in accordance with various embodiments of the present disclosure.

FIG. 2 illustrates prediction results by Scan Point/Site for DarkNet-19, in

accordance with various embodiments of the present disclosure.

FIG. 3 illustrates prediction results by Scan Point/Site for ShrapML, in accordance with various embodiments of the present disclosure.

FIG. 4 illustrates a main or home screen of a user interface of an application (app), in accordance with various embodiments of the present disclosure.

FIG. 5 illustrates a main or home screen of a user interface of an application, in accordance with various embodiments of the present disclosure.

FIG. 6 illustrates a recording screen of a user interface of an application, in accordance with various embodiments of the present disclosure.

FIG. 7 illustrates a saved screen of a user interface of an application, in accordance with various embodiments of the present disclosure.

FIG. 8 illustrates a video processing screen of a user interface of an application, in accordance with various embodiments of the present disclosure.

FIG. 9 illustrates a select crop mask screen of a user interface of an application, in accordance with various embodiments of the present disclosure.

FIG. 10 illustrates a preview mask screen of a user interface of an application, in accordance with various embodiments of the present disclosure.

FIG. 11A and FIG. 11B illustrate select scan site screens of a user interface of an application, in accordance with various embodiments of the present disclosure.

FIG. 12 illustrates a video processing screen of a user interface of an application, in accordance with various embodiments of the present disclosure.

FIG. 13 illustrates an example of a folder 1300 containing processed ultrasound video frames, in accordance with various embodiments of the present disclosure.

FIG. 14 illustrates a predictions screen of a user interface of an application (app), in accordance with various embodiments of the present disclosure.

FIG. 15 illustrates a predictions screen of a user interface of an application (app), in accordance with various embodiments of the present disclosure.

FIG. 16 illustrates a screen of a user interface of an application to show frames during processing, in accordance with various embodiments of the present disclosure.

FIG. 17 is a system diagram of an environment for an injury prediction system, in accordance with various embodiments of the present disclosure.

FIG. 18 is a system diagram of a cloud computing environment for an injury prediction system, in accordance with various embodiments of the present disclosure.

FIG. 19 is a flow diagram for predicting internal trauma of a patient using ultrasound and trained AI models, in accordance with various embodiments of the present disclosure.

### DETAILED DESCRIPTION

In accordance with the disclosure, there are provided certain system and method embodiments to diagnose injuries in canines or other patients, Artificial Intelligent (AI) ultrasound image interpretation models are used to simplify MWD care and care of other canines on the battlefield and in other working environments. As described in this disclosure, AI driven software is used for capturing and interpreting images, allowing human medical personal to quickly and accurately triage MWDs. AI models with multiple architectures are trained using ultrasound images collected in MWDs for this application as well as cadaver tissue. Performance was shown to be high across all Global FAST scan points, a triage exam looking for free fluid/air in the thoracic and abdominal.

Through capturing images in a wider range of dog breeds, the underlying AI-based system, device and methodologies presented herein can be expanded beyond just MWDs into a wider civilian veterinary care market.

Military working dogs (MWDs) are essential for military operations in a wide range of missions. With this pivotal role, MWDs can become casualties requiring specialized veterinary care that may not always be available far forward on the battlefield. Some injuries such as pneumothorax, hemothorax, or abdominal hemorrhage can be diagnosed using point of care ultrasound (POCUS) such as the GlobalFAST® exam. This presents a unique opportunity for artificial intelligence (AI) to aid in the interpretation of ultrasound images. In this disclosure, deep learning classification neural networks, CNN or other AI provide for POCUS assessment in MWDs.

Therefore, in accordance with the disclosure, there are provided certain system and method embodiments to automatically predict an internal trauma of a patient, such as MWDs, canines or other patients.

In accordance with certain embodiments a method for automatically predicting an internal trauma of a patient is provided and described, the method including processing point of care ultrasound (POCUS) images of the patient, each POCUS image associated with a scan site of the patient, in accordance with one or more selected scan sites of the patient and augmentation settings to generate processed POCUS images of the patient; and interpreting the processed POCUS images of the patient associated with the one or more selected scan sites and augmentation settings using one or more trained AI models to automatically generate a predicted internal trauma injury result at a selected scan site of the patient.

In accordance with certain embodiments a system for automatically predicting an internal trauma of a patient is provided and described, the system including: a controller; a processing circuit controlled by the controller and configured to process a plurality of point of care ultrasound (POCUS) images of the patient in accordance with one or more selected scan sites of the patient and augmentation settings to generate processed POCUS images of the patient, each POCUS image associated with a scan site of the patient; and a prediction circuit controlled by the controller and configured to interpret the processed POCUS images of the patient associated with the one or more selected scan sites and augmentation settings using one or more trained AI models to automatically generate a predicted internal trauma injury result at a selected scan site of the patient, where the controller controls the prediction circuit to communicate the predicted internal trauma injury result through a user interface of the system controlled by the controller.

Methods: Images were collected in five MWDs under general anesthesia or deep sedation for all scan points in the GlobalFAST® exam. For representative injuries, a cadaver model was used from which positive and negative injury images were captured. A total of 327 ultrasound clips were captured and split across scan points for training three different AI network architectures: MobileNetV2, DarkNet-19, and ShrapML. Gradient class activation mapping (GradCAM) overlays were generated for representative images to better explain AI predictions.

Results: Performance of AI models reached over 82% accuracy for all scan points. The model with the highest performance was trained with the MobileNetV2 network for the cystocolic scan point achieving 99.8% accuracy. Across all trained networks the diaphragmatic hepatorenal scan point had the best overall performance. However, GradCAM overlays showed that the models with highest accuracy, like MobileNetV2, were not always identifying relevant features. Conversely, the GradCAM heatmaps for ShrapML show general agreement with regions most indicative of fluid accumulation.

Discussion: Overall, the AI models developed can automate POCUS predictions in MWDs. Preliminarily, ShrapML had the strongest performance and prediction rate paired with accurately tracking fluid accumulation sites, making it the most suitable option for integration and real-time deployment with ultrasound systems. Integration of this technology with imaging technologies expands use of POCUS-based triage of MWDs.

Towards addressing this critical capability gap for canine and human casualties on the future battlefield, AI is utilized to automate medical triage image interpretation (Latif et al., 2019; Liu et al., 2020). AI for image interpretation may rely on deep convolutional neural network (CNN) or other AI models containing millions of trainable parameters to extract features from images for making categorical predictions (Liu et al., 2019; Komatsu et al., 2021). For medical applications, AI is used for tumor detection (Chiang et al., 2019; Song et al., 2023), COVID-19 diagnosis (Diaz-Escobar et al., 2021; Gil-Rodríguez et al., 2022), and obstetric ultrasound applications (Baumgartner et al., 2017; Iriani Sapitri et al., 2023). In addition, AI is applied to interpret radiographs in thoracic (Banzato et al., 2021; Müller et al., 2022), cardiac (Li et al., 2020; Kim et al., 2022), and orthopedic (McEvoy et al., 2021) settings. A previously developed ultrasound image AI interpretation model was used for detecting shrapnel in tissue, termed ShrapML (Boice et al., 2022; Snider et al., 2022). This work is expanded to the enhanced FAST (eFAST) exam used for human emergency triage applications (Hernandez-Torres et al., 2023), resulting in different AI models for detecting pneumothorax, hemothorax, and abdominal hemorrhage injuries in tissue phantom image sets.

In the present disclosure, AI image interpretation models are trained on canine image datasets and are able to automatically identify injuries at each POCUS scan point. By utilizing this approach, the skill threshold for POCUS interpretation will be lowered so that this critical triage task can be available at early echelons of care where emergency intervention is most needed for MWDs and other types of patients.

### MATERIALS AND METHODS

### Imaging Protocol

Research was conducted in compliance with the Animal Welfare Act, the implementing Animal Welfare regulations, and the principles of the Guide for the Care and Use for Laboratory Animals. The Institutional Animal Care and Use Committee at the Department of Defense Military Working Dog Veterinary Services approved all research conducted in this study. The facility where this research was conducted is fully accredited by the AAALAC International. The POCUS protocol used mirrored the GlobalFAST® procedure in a total of five (1.5 to 10 years old) healthy canine subjects (20 to 55 kgs weight) under general anesthesia or deep sedation for other medical procedures, as prescribed by the attending veterinarian. Ultrasound (US) clips were collected in 8 scan points (Table 1) using a C11 transducer (Fujifilm, Bothell, WA, USA) with a Sonosite Edge ultrasound system (Fujifilm, Bothell, WA, USA). The subject was positioned in right lateral, left lateral, sternal or dorsal recumbency for ease of access to each scan point. A minimum of three 15 second clips were collected at each scan point with the probe orientation held in the coronal plane for the first 6 seconds and then rotated to the transverse plane for the remainder of each clip. All clips collected from the live subjects were used as baseline (negative for injury) data. The same scanning protocol was used to obtain US imaging data from a cadaver canine model. A total of five frozen cadavers (Skulls Unlimited, Oklahoma City, OK, USA) were received and stored at −20° C. until ready for use. Once thawed, an endotracheal tube (Mckesson Medical-Surgical, Irving, TX, USA) was placed into the trachea of each subject and secured to a bag valve mask (EMS Safety Services, Eugene, OR, USA) for ventilation. At this time thoracic and abdominal CT scans (Toshiba Aquilion CT Scanner, Cannon Medical Systems, Tustin, CA, USA) were collected to identify any pre-existing injuries. Then, data was collected at each scan point, using the same protocol as the live subjects. After collecting the first round of data, if the subject was positive for any injury e.g. a pneumothorax, a needle decompression was performed to remove air and obtain a negative scan. Another round of data was collected with the scan points that were negative for injury. Next, controlled injuries were performed by adding blood or saline to the pleural space (up to 300 mL) or the abdomen (up to 400 mL) for a final round of positive injury image collection in the cadaver subjects.

### Preprocessing Images

All clips were exported from the US machine as MP4 format and then renamed to reflect the scan point, subject ID, and recumbency of each subject. Frames were extracted from each clip using ffmpeg tool, via a Ruby script, and then sorted by positive or negative for injury by scan point. Each frame was then cropped to remove the user interface information from the US system and the images were resized to 512×512 pixels. Additional steps were taken with images collected at the chest-tube or thoracic sites, to recreate M-mode images. For example, clips were processed to extract a pixel-wide image over time for visualizing the lung-pleura interface movement. These custom M-mode images were then cropped and resized to 512×512 as well.

Before images were ready for training, they were augmented to prevent model overfitting and improve performance. While data augmentation is useful to prevent overfitting, it can result in poor model performance and more computationally intensive training if not setup optimally for the application (Xu et al., 2020). A representative image was chosen from each scan point, including M-mode reconstructions, to match histogram values across all the other images using the “imhistmatch” function by MATLAB (MathWorks, Natick, MA, USA). Then, contrast and brightness were randomly adjusted by ±20% to add training noise using the “jitterColorHSV” function by MATLAB. Both MATLAB functions were applied to all images for every scan point using Image Batch Processor on MATLAB. Augmented US images were imported at a 512×512×3 image size and were randomly assigned to training, validation or testing datasets at a 70:15:15 ratio. Image sets were set up so that an even number of positive or negative images were selected in each dataset for each split. Next, training images were augmented randomly by affine transformations: random scaling, random X and Y reflections, random rotation, random X and Y shear, and random X and Y translation. However, for the CTS two-dimensional M-mode scan point only X reflection and translation affine transformations were applied given how these images were constructed. Due to DH scan point images being unable to train with all augmentations (data not shown), only reflection and translation augmentations were applied for both the X and Y direction.

### Training AI Models

Three different AI models were evaluated for this application-MobileNetV2, DarkNet-19, and ShrapML. MobileNetV2 has 53 convolutional layers, 3.5 million parameters, and was optimized for use on mobile devices. This architecture is able to perform at the highest accuracy for identifying shrapnel in a custom tissue phantom. The second-best performing architecture, DarkNet-19, has 19 convolutional layers, 20.8 million parameters, and utilizes global average pooling for making predictions. The last model used, ShrapML, was purpose built and Bayesian optimized for identifying shrapnel in ultrasound images at a high accuracy and much more rapid than conventional models. In addition, ShrapML is successful at identifying pneumothorax, hemothorax, and abdominal hemorrhage injuries in eFAST images captured in human tissue phantom models (Hernandez-Torres et al., 2023). ShrapML consists of 8 convolutional layers with only 430,000 trainable parameters.

Training for all scan points consisted of a learning rate of 0.001 with a batch size of 32 images and RMSprop (root mean squared propagation) as the optimizer. A maximum of 100 epochs was allowed for training with a validation patience of 5 epochs if the overall validation loss did not improve. The model with the lowest validation loss was selected for use with blind predictions. All training was performed using MATLAB R2022b run on a Microsoft Windows workstation with a NVIDIA Geforce RTX 3090 Ti 24 Gb VRAM graphics card, Intel i9-12900k and 64 GB RAM.

### Performance Metrics

Testing image sets were used to assess blind performance in multiple ways. First, confusion matrices were generated to categorize prediction as either true positive (TP), true negative (TN), false positive (FP), or false negative (FN) results. These results were used to generate performance metrics for accuracy (1), recall (2), precision (3), specificity (4), and F1 (5) scores using these respective formulas.

$$Accuracy = \frac{( TP + TN )}{( TP + TN + FP + FN )} \qquad ( 1 )$$

$$Precision = \frac{TP}{TP + FP} \qquad ( 2 )$$

$$Recall = \frac{TP}{TP + FN} \qquad ( 3 )$$

$$Specificity = \frac{TN}{TN + FP} \qquad ( 4 )$$

$$F 1 score = \frac{2 \times Precision \times Recall}{Precision + Recall} \qquad ( 5 )$$

Then, receiver operating characteristic (ROC) plots were constructed to further classify performance for a number of confidence thresholds for the predictions. ROC plots were used to calculate the area under the ROC curve or AUROC, which tells you how well the model differentiates between categories. Next, inference time for test image predictions were quantified for each trained model to assess differences in computational efficiency of the three different AI models used. Lastly, Gradient-weighted Class Activation Mapping (GradCAM) overlays were generated for test predictions to highlight the regions of images where the AI predictions were focused. These were used as an explainable-AI methodology to verify the AI models were accurately tracking the image regions where injury differences were present.

### RESULTS

### MobileNetV2

MobileNetV2 model was successfully trained for each POCUS scan point,

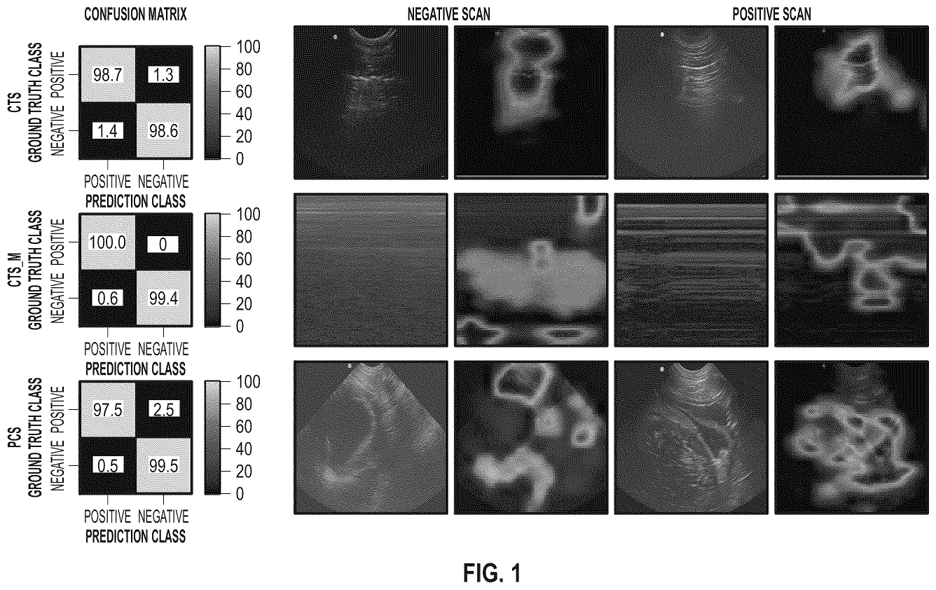

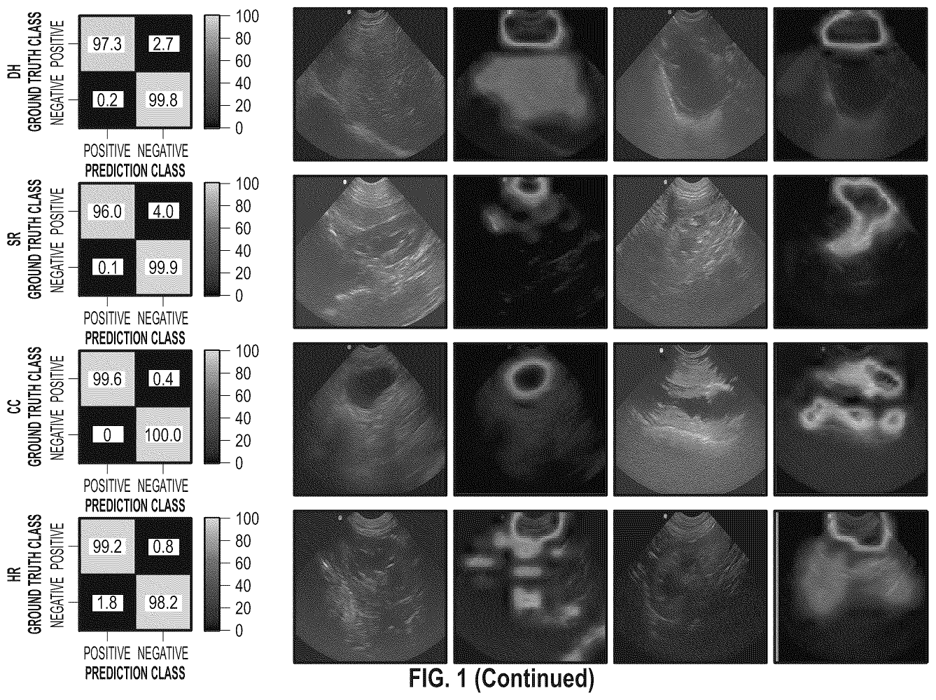

with an average accuracy across all locations of 98.8% (see Table 2). In addition, strong performance was evident for other conventional metrics across each POCUS scan point. However, upon closer inspection using GradCAM mask overlays, the MobileNetV2 trained model was not always properly tracking the injury site, but instead was focused on image artifacts that will likely not be consistent for additional canine subjects not included in the current datasets (FIG. 1). CTS scan sites for both M- and B-mode were accurately tracking injuries, other scan sites such as HR, DH, and SR were not tracking correctly. Average inference times across all MobileNetV2 scan site models was 6.21 ms per prediction.

### DarkNet-19

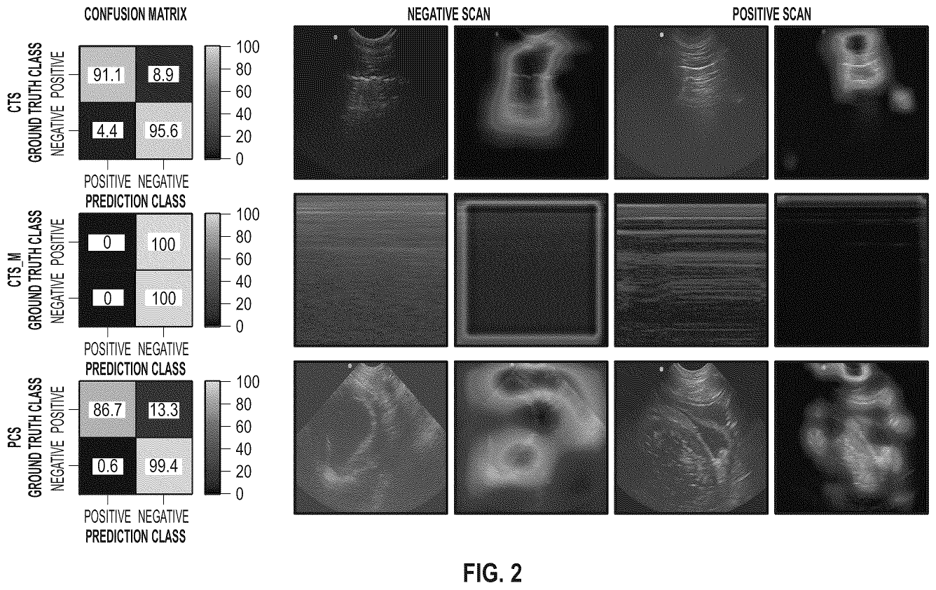

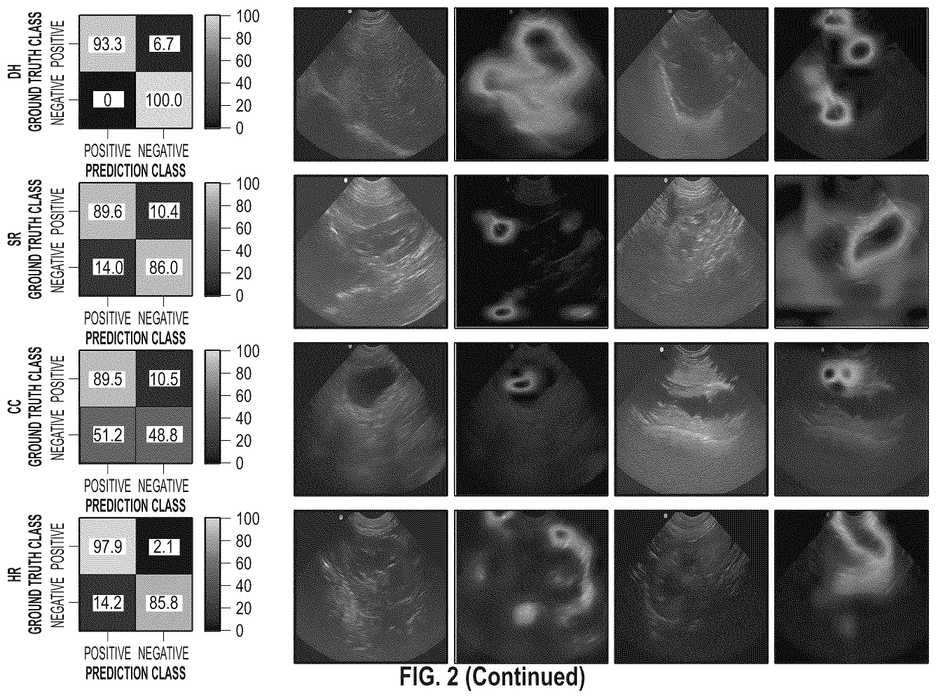

The DarkNet-19 models had similar inference speeds compared to MobileNetV2 at 5.93 ms per prediction, but overall performance was reduced for a number of the scan sites, resulting in an average accuracy across all scan points of 86.4% (Table 3). Certain scan points like chest-tube M-mode images resulted only in predictions of negative (TN or FN) and the GradCAM overlays identified no obvious tracked features (FIG. 2). While this was the worst performing dataset trained against, the Cystocolic scan site was also only at 69.2% accuracy. While performance was reduced compared to MobileNetV2 across nearly all metrics, the GradCAM overlays were more accurately tracking image features more consistent with locations where free fluid was or could be identified. These results indicated that while performance was overall reduced for DarkNet-19, the predictions were more often tracking the proper image features. More images and subject variability may improve on training performance.

### ShrapML

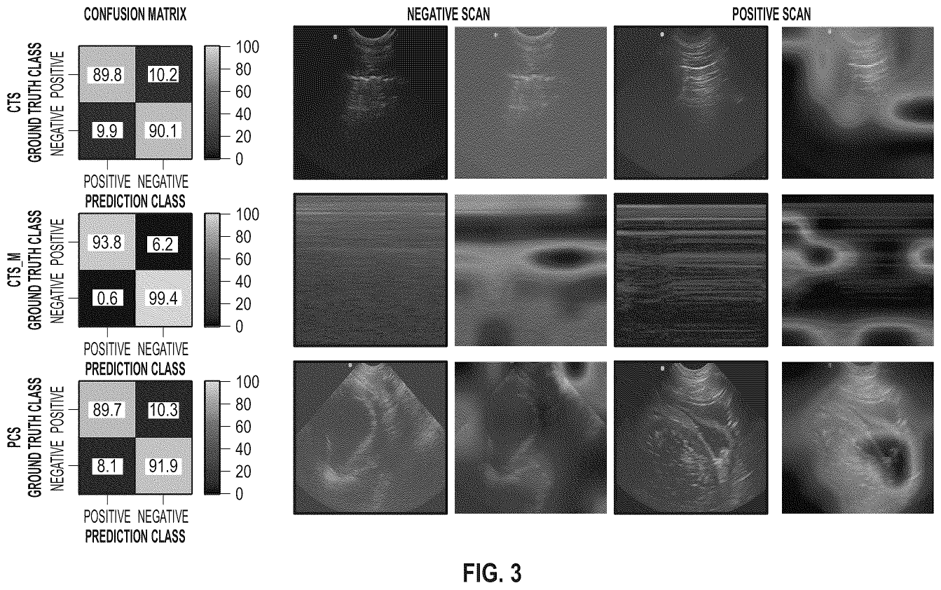

The last model evaluated was ShrapML, which resulted in an accuracy across all scan sites of 93.4% (Table 4). Unlike DarkNet-19, no trained model resulted in an instance of 100% positive or negative guesses. However, performance metrics were consistently worse than MobileNetV2. Given the smaller model size of ShrapML, the inference times were much quicker compared to the other models with prediction rates at an average of 3.43 ms per image. GradCAM overlays more closely resembled DarkNet-19 in that many of the heat map intensity points were focused on regions where free fluid was likely to be found or near organs present in the ultrasound scan (FIG. 3), except for the HR site. Overall, ShrapML was successful at performing similarly well to these large network structures for this GlobalFAST application, model overfitting was less evident in the results, and overall prediction speed outperformed the other models tested.

A summary table of average performance metrics for each scan site across all three model architectures is shown in Table 5.

### DISCUSSION

Medical imaging-based triage is critical for both human and veterinary emergency medicine to identify issues early on and ensure resources are properly distributed. In remote or military medicine situations, the lack of skilled personnel makes imaging based-triage less relied upon, but AI prediction models can simplify this for the end user. Here, a focus was on the POCUS procedure GlobalFAST®, a widely used triage exam to look for abdominal or thoracic free fluid in injured dogs. The AI models shown in this work can automate predictions for ultrasound results when properly tuned for the application.

Three different AI architectures were evaluated to see which was capable of being trained to distinguish positive injury cases from baseline images. While all models are generally successful at being trained for these applications, strong test performance may not indicate properly trained models. For instance, MobileNetV2 had the highest accuracy, but heat map overlays indicating where the AI was focused were not tracking proper image locations. Model overfit was combatted with the various image augmentation techniques used for the training, but this was insufficient to mimic proper subject variability to create a more robust model for this architecture. This issue was less evident for the other two model architectures, highlighting the importance of AI model selection and validation on ultrasound image applications such as this. However, without more subjects and the variability that those bring, it is hard to fully verify if the developed DarkNet-19 or ShrapML models are suitable. Preliminarily, ShrapML had the strongest performance and prediction rate, making it the most suitable going forward as well as eventual integration for real-time deployment with ultrasound machines.

Focusing on the various scan points in the used POCUS exam, there were obvious differences in the AI model training. Training image sets were not equally sized, but that did not correlate to what scan sites performed the best. The DH site was the overall strongest performing site across all performance metrics. However, this could be due to this scan site having the largest difference between live and cadaveric tissue resulting in a well-trained model. In addition, less augmentation steps were used for this site due to training issues using all affine transformations. More images are needed to address this issue from a wider range of subjects. CTS and HR views also performed well across the three models trained. Worst performing was the M-mode reconstructed chest-tube images which could be influenced by the minimal training data used for this model, and thus may be improved with more training data. The CC site was also a lower performing scan site even though more than 10,000 images were used in the training dataset. However, this is mostly influenced by DarkNet-19 having lower performance for this scan site while the other two models had accuracies greater than 96%. Overall, each scan site for this POCUS application was successful as an input for an injury prediction model.

### CONCLUSION

Artificial intelligence has the potential to simplify triage and injury diagnosis for emergency veterinary medicine. The results shown in this work highlight how AI can be used for automating US detection of intrabdominal and intrathoracic injury detection for veterinary applications. Each scan point reached greater than 80% injury detection accuracy, with most surpassing 90% accuracy. These models provide for real-time integration with ultrasound devices allowing for early detection of thoracic and abdominal injuries for military working dogs and other canine trauma situations. This will lower the skill threshold for medical imaging-based triage so that these techniques can be widely used.

Referring now to Table 1 below, example Scan Point/Site descriptions of the POCUS imaging protocol is provided.

TABLE 1

| Scan | | |
|---|---|---|
| Point/Site | Abbreviation | Description |
| Bilateral | CTS | Longitudinal plane on both sides of the |
| Chest | | chest perpendicular to the ribs at the 7^{th} to |
| Tube Site | | 9^{th} intercostal space. |
| Bilateral | PCS | Longitudinal and transverse planes on |
| Pericardial | | each side of the chest between the 5^{th} |
| Site | | and 6^{th} intercostal spaces over the heart. |
| Diaphragmatic | DH | Subxiphoid view for visualization of the |
| Hepatic | | pleural and pericardial spaces beyond |
| | | the diaphragm to evaluate |
| | | hepatodiaphragmatic interface, |
| | | gallbladder region, and pericardial sac. |
| Splenorenal | SR | Left flank view to assess the splenorenal |
| | | interface and areas between the spleen |
| | | and body wall |
| Cystocolic | CC | Midline view to assess the apex of the |
| | | bladder |
| Hepatorenal | HR | Right flank view to assess the hepatorenal |
| | | interface and areas between the spleen |
| | | and body wall |

Table 2 below provides a summary of performance metrics for the MobileNetV2 AI convolutional neural network architecture

TABLE 2

| Metric | CTS | CTS M-mode | PCS | DH | SR | CC | HR | Average |
|---|---|---|---|---|---|---|---|---|
| Accuracy | 0.987 | 0.997 | 0.985 | 0.986 | 0.979 | 0.998 | 0.987 | 0.988 |
| Precision | 0.986 | 0.994 | 0.995 | 0.998 | 0.999 | 1.000 | 0.982 | 0.995 |
| Recall | 0.987 | 1.000 | 0.976 | 0.973 | 0.960 | 0.996 | 0.992 | 0.980 |
| Specificity | 0.986 | 0.994 | 0.995 | 0.998 | 0.999 | 1.000 | 0.982 | 0.995 |
| F1 Score | 0.987 | 0.997 | 0.985 | 0.985 | 0.979 | 0.998 | 0.987 | 0.987 |
| AUROC | 0.999 | 1.000 | 1.000 | 1.000 | 0.999 | 1.000 | 0.999 | 1.000 |
| Inference | 6.22 | 7.67 | 5.59 | 5.58 | 6.64 | 6.06 | 6.57 | 6.21 |
| Time | | | | | | | | |
| (ms/image) | | | | | | | | |

Referring now to the drawings, where like reference numerals designate identical or corresponding parts throughout the several views, the following description relates to a dedicated system and method for automatically predicting an internal trauma of a patient.

FIG. 1 illustrates prediction results by Scan Point/Site for MobileNetV2. Results for each scan site showing in column 1 confusion matrix test prediction results, negative representative images in columns 2-3 and positive representative images in columns 4-5 without and with the GradCAM overlay. Regions in the images with high relevance to model predictions have red-yellow overlays, while those of lower relevance have blue-green overlays.

Table 3 below provides a summary of performance metrics for DarkNet-19 AI convolutional neural network architecture. FIG. 2 illustrates prediction results by Scan Point/Site for DarkNet-19. Results for each scan site showing in column 1 confusion matrix test prediction results, negative representative images in columns 2-3 and positive representative images in columns 4-5 without and with the GradCAM overlay. Regions in the images with high relevance to model predictions have red-yellow overlays, while those of lower relevance have blue-green overlays.

TABLE 3

| Metric | CTS | CTS M-mode | PCS | DH | SR | CC | HR | Average |
|---|---|---|---|---|---|---|---|---|
| Accuracy | 0.933 | 0.500 | 0.930 | 0.967 | 0.878 | 0.692 | 0.919 | 0.864 |
| Precision | 0.954 | | 0.993 | 1.000 | 0.865 | 0.636 | 0.873 | 0.844 |
| Recall | 0.911 | 0.000 | 0.867 | 0.933 | 0.896 | 0.895 | 0.979 | 0.926 |
| Specificity | 0.956 | 1.000 | 0.994 | 1.000 | 0.860 | 0.488 | 0.858 | 0.801 |
| F1 Score | 0.932 | | 0.926 | 0.966 | 0.880 | 0.744 | 0.923 | 0.878 |
| AUROC | 0.984 | 0.575 | 0.992 | 0.999 | 0.953 | 0.737 | 0.988 | 0.92 |
| Inference Time | 6.32 | 8.73 | 5.53 | 5.61 | 5.86 | 6.17 | 6.07 | 5.93 |
| (ms/image) | | | | | | | | |

Table 4 below provides a summary of performance metrics for ShrapML AI convolutional neural network architecture. FIG. 3 illustrates prediction results by Scan Point/Site for ShrapML. Results for each scan site showing in column 1 confusion matrix test prediction results, negative representative images in columns 2-3 and positive representative images in columns 4-5 without and with the GradCAM overlay. Regions in the images with high relevance to model predictions have red-yellow overlays, while those of lower relevance have blue-green overlays.

TABLE 4

| Metric | CTS | CTS M-mode | PCS | DH | SR | CC | HR | Average |
|---|---|---|---|---|---|---|---|---|
| Accuracy | 0.900 | 0.966 | 0.908 | 0.989 | 0.861 | 0.965 | 0.950 | 0.934 |
| Precision | 0.901 | 0.994 | 0.917 | 0.993 | 0.806 | 0.967 | 0.977 | 0.936 |
| Recall | 0.898 | 0.938 | 0.897 | 0.984 | 0.950 | 0.963 | 0.921 | 0.936 |
| Specificity | 0.901 | 0.994 | 0.919 | 0.993 | 0.772 | 0.967 | 0.978 | 0.932 |
| F1 Score | 0.900 | 0.965 | 0.907 | 0.988 | 0.872 | 0.965 | 0.948 | 0.935 |
| AUROC | 0.961 | 0.998 | 0.97 | 0.999 | 0.928 | 0.995 | 0.988 | 0.977 |
| Inference Time | 5.72 | 3.78 | 2.63 | 2.68 | 3.31 | 2.83 | 3.05 | 3.43 |
| (ms/image) | | | | | | | | |

Table 5 below provides a summary of performance metrics for each POCUS scan point/site.

TABLE 5

| | CTS | CTS M-Mode | PCS | DH | SR | CC | HR |
|---|---|---|---|---|---|---|---|
| Accuracy | 93.98% | 82.11% | 94.12% | 98.02% | 90.61% | 88.49% | 95.18% |
| Precision | 94.69% | 99.42% | 96.83% | 99.69% | 89.00% | 86.76% | 94.42% |
| Recall | 93.19% | 64.60% | 91.32% | 96.35% | 93.52% | 95.15% | 96.41% |
| Specificity | 94.77% | 99.62% | 96.92% | 99.70% | 87.69% | 81.82% | 93.95% |
| F1 Score | 93.92% | 98.11% | 93.93% | 97.98% | 91.04% | 90.22% | 95.29% |
| Number of | 23,305 | 1,652 | 16,380 | 11,340 | 9,455 | 10,080 | 9,455 |
| Training | | | | | | | |
| Images | | | | | | | |

Preliminary data was collected from two groups: healthy MWDs for baseline image capture and a cadaver canine injury model for baseline and injury images. This was curated and used to train AI models using three architectures at six POCUS scan points, as shown below in Table 6 below, in which preliminary data used to train example AI models are shown. Further data and datasets will be used to further refine AI models.

TABLE 6

| CNN Model | CTS | CTS M-Mode | PCS | DH | SR | CC | HR |
|---|---|---|---|---|---|---|---|
| ShrapML | 90.0% | 96.6% | 90.8% | 98.9% | 86.1% | 96.5% | 95.0% |
| DarkNet-19 | 93.3% | 50.0% | 93.0% | 96.7% | 87.8% | 69.2% | 91.9% |
| MobileNetV2 | 98.7% | 99.7% | 98.5% | 98.6% | 97.9% | 99.8% | 98.7% |
| Average | 94.0% | 82.1% | 94.1% | 98.0% | 90.6% | 88.5% | 95.2% |

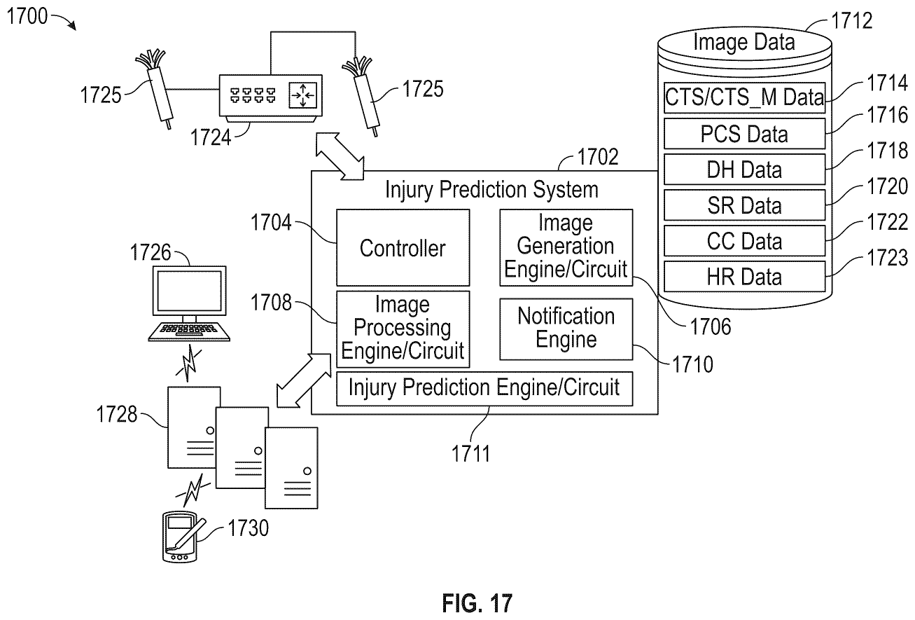

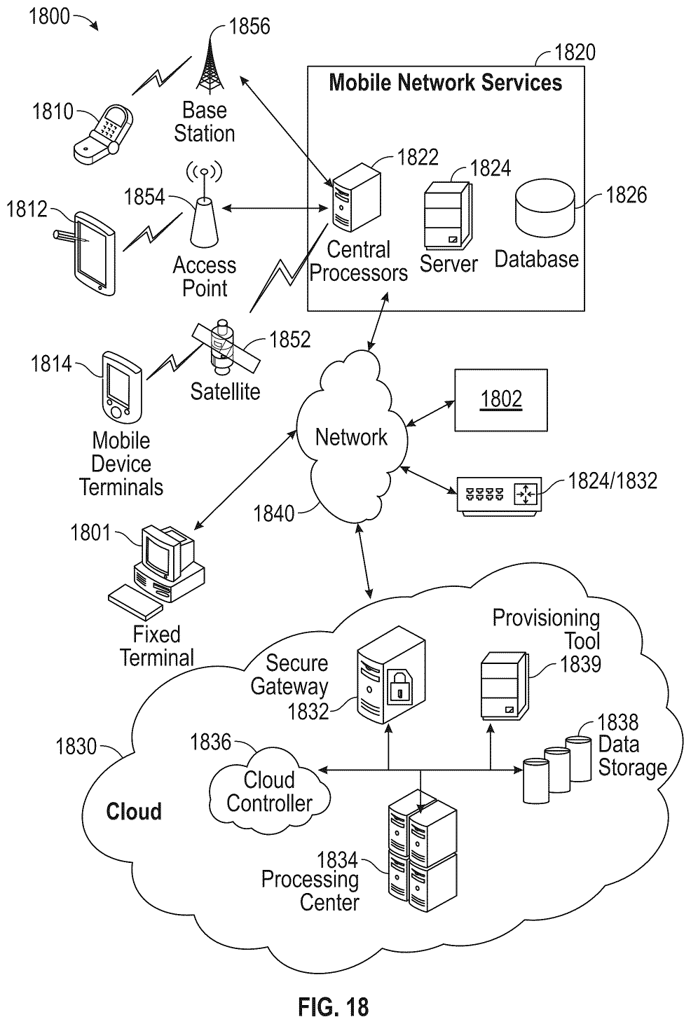

In accordance with certain embodiments, an application or app of a system allows an internal trauma of a patient to be automatically predicted using ultrasound technology. Such an application may running on a portable device, such as a handheld ultrasound system (not doppler signal based) that is easily portable and allows a patient's internal trauma injuries to be predicted in the field. Conversely, an application may also be employed within a system, such as a cloud-based system, that allows ultrasound scans conducted in the field to be accessed at a later time. In either case, the application allows a user to readily interface with the system to predict traumatic injuries of a patient. FIGS. 17 and 18 illustrate a system block diagram for automatic prediction of internal trauma and a cloud-based system, respectively, that may interface with such a system and are discussed further below.

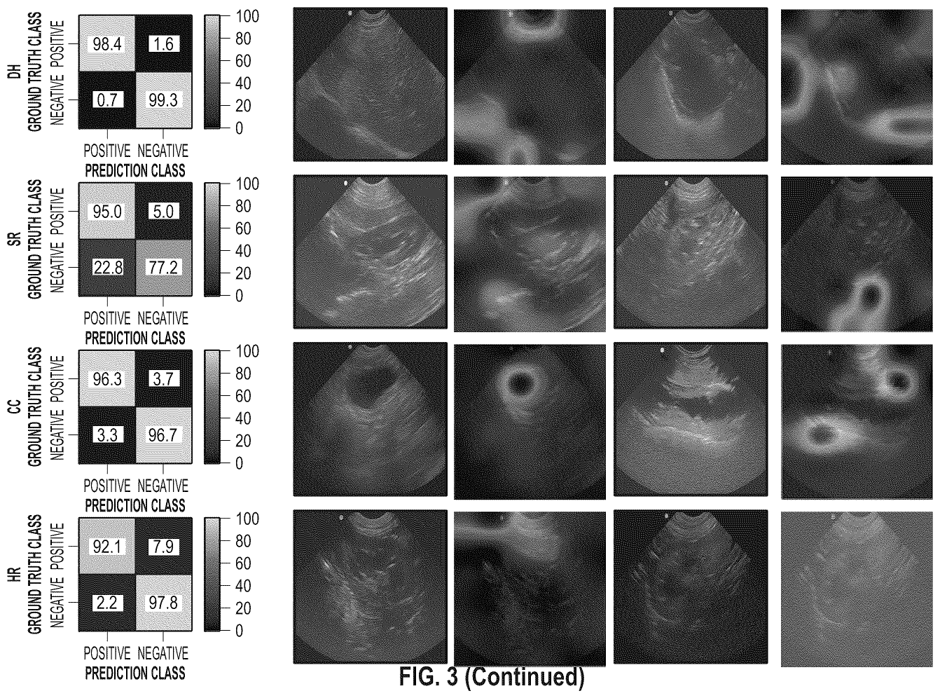

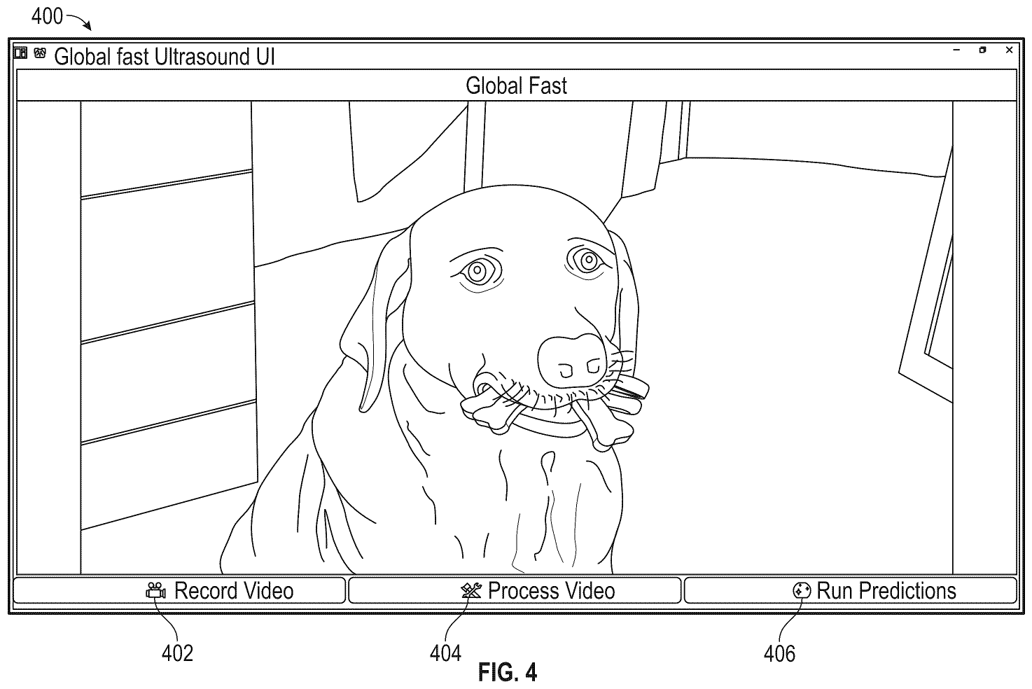

Referring now to FIG. 4, a main or home screen of a user interface 400, such as a Global Fast Ultrasound UI, a graphical user interface (GUI), is shown. Options are presented to a user of the application, illustrated by Record Video button 402, Process Video button 404 and Run Predictions button 406. These actions represent methodologies open to the user of the app through use of the app supported by a system for prediction of traumatic internal injuries of a patient.

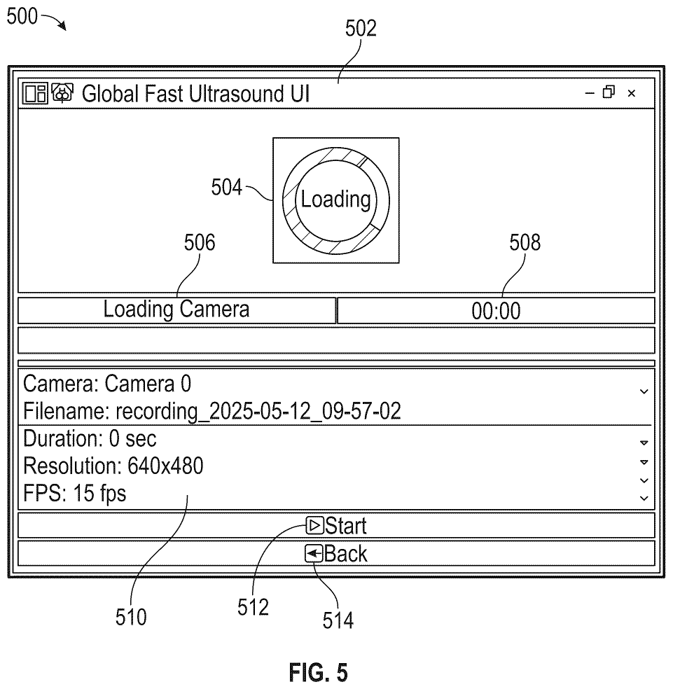

Upon opening, the app will search for all available cameras and then search all available resolutions and frame rates for each camera. This may take some time, and the user will see the loading screen 500 of GlobalFast Ultrasound UI 502 of FIG. 5 until this is completed. This searching helps make sure that unsupported resolutions are not selected, which can cause silent crashes and corrupted videos in the system. This loading screen is also shown (for a shorter time) when switching cameras or changing camera settings, this is because the app is reinitializing the camera feed with the new camera. Without this step, an error and/or crash can be caused by opening multiple camera feeds at once.

The loading screen has a loading graphic 504, a button 506 that communicates that a camera feed is being loaded and a button 508 that communicates for how long. The drop down screens in buttons 510 communication to a user of the app what camera is being loaded with which file name of a recording and the duration, resolution and how many frames per second (FPS). The loading screen further has start button 512 and back button 514 as shown.

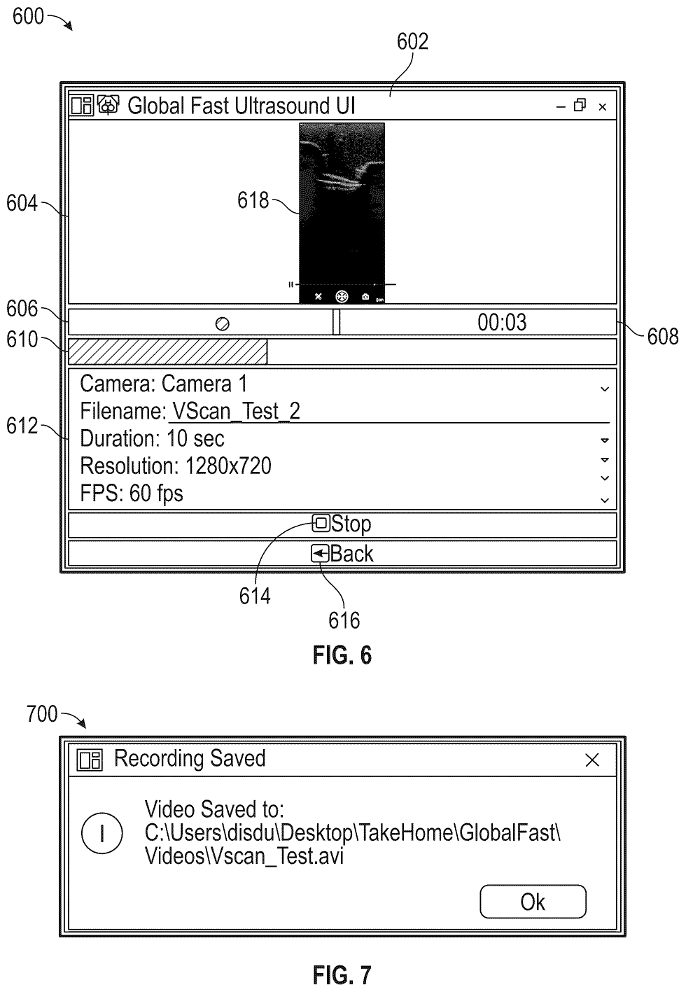

Referring now to recording screen 600 of GlobalFast Ultrasound UI 602 of the app in FIG. 6, after setting up the camera settings using button 612 (in this case Camera 1 is selected), a user of the app may push the start button 606 to begin recording. The user will see a progress bar 610 and the time will increment while recording, as shown in the time elapsed displayed in button 608. In certain embodiments, if the “Duration” field of button 612 is set to 0, the video will record until the user stops the recording by selecting stop button 606. Following recording of the ultrasound scan, the recording is saved. The Recording Saved message 700 of FIG. 7 may be shown to the user via the User Interface when the video recording completes.

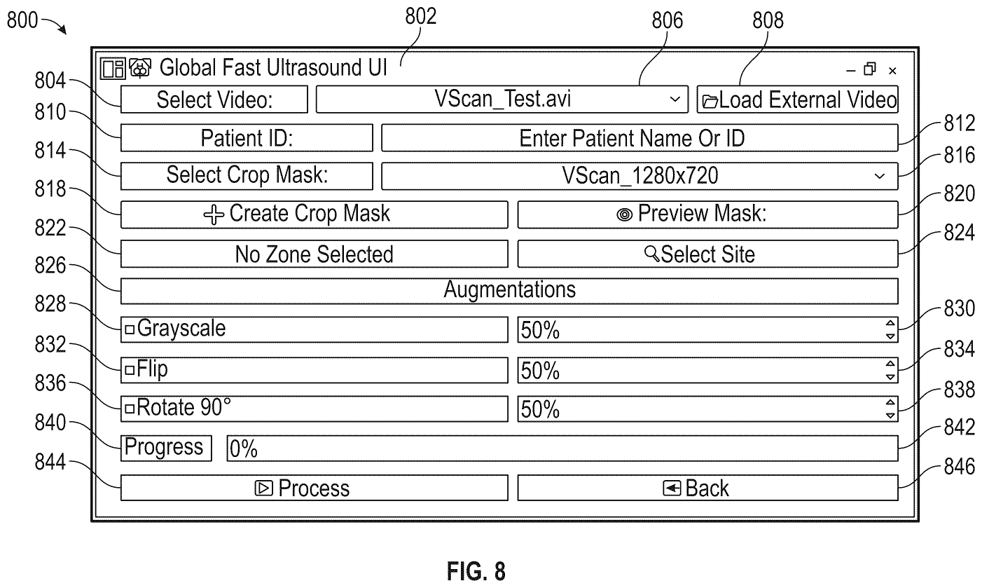

Referring back to FIG. 4, the Process Video button 404 of FIG. 4 may next be selected in order to process the ultrasound images captured during the US scan of the patient. FIG. 8 illustrates a video processing screen 800 that is shown to the user of the app via the GUI when the Process Video button 404 is activated (chosen) by the app user. Video Processing screen 800 of a Global Fast Ultrasound UI 802 has a number of fields as shown. The Select Video button 804 allows the user to select a video in video dropdown button 806; in this example, the user has selected VScan Test.avi.

The Select Video dropdown button 806 is automatically populated with videos from the app's internal folder, but the user has the option to open the file explorer and load an external video, which will be added to the dropdown menu of button 808, with “Load External Video” displayed.

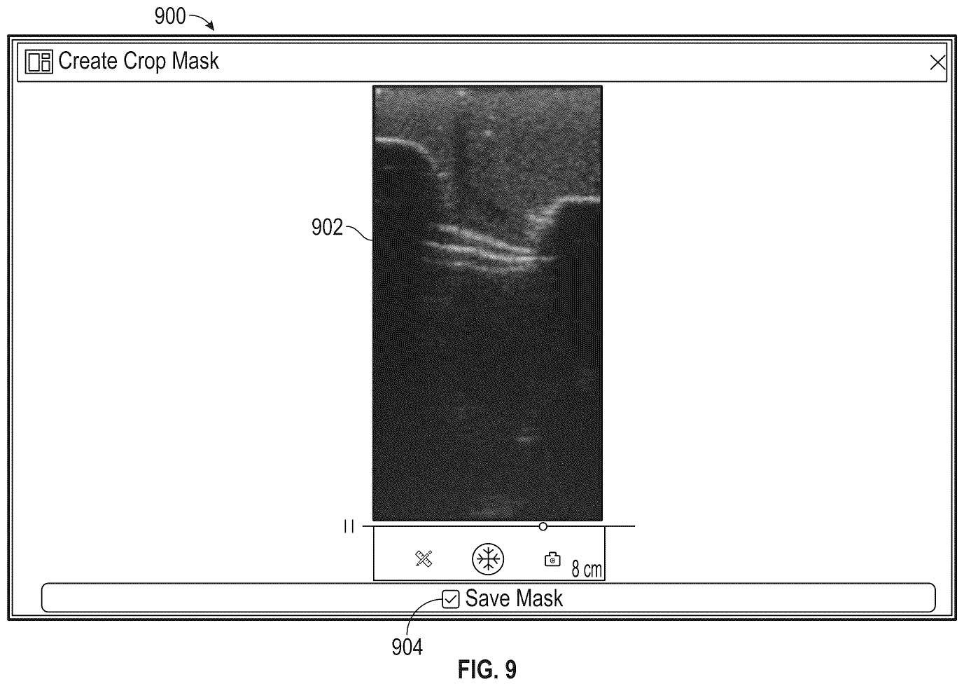

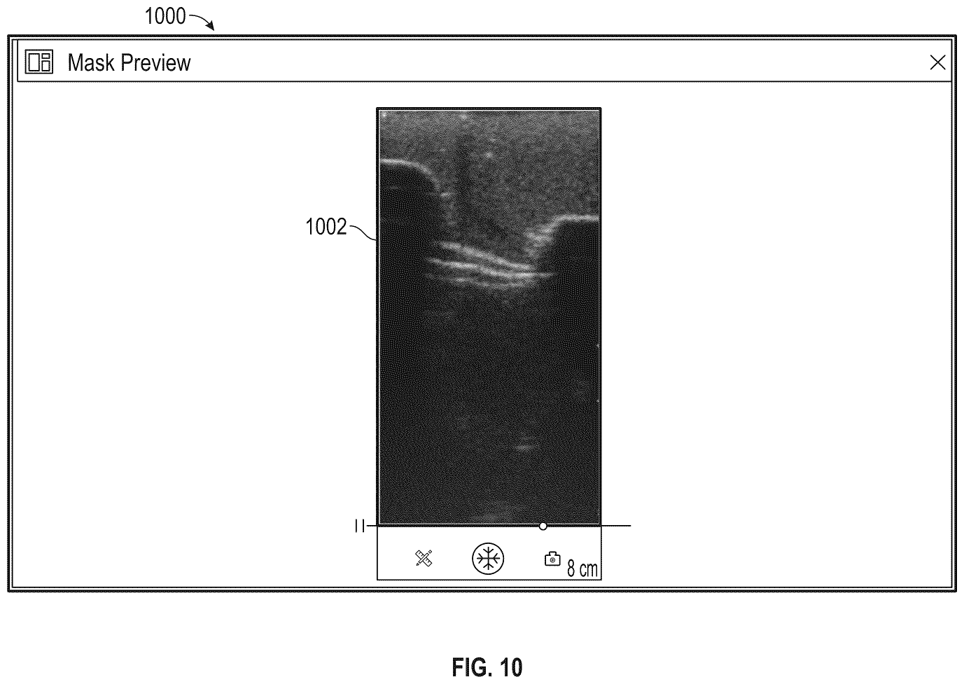

The Select Crop Mask: button 814 may also be populated with internally stored cropping masks, but the user has to option to define their own, as shown in FIG. 9. The preview mask button 820 will show the currently selected mask overlaid on the first frame of the currently selected video, VScan_1280×720, in this example. Referring to 900 of FIG. 9, a user may create custom cropping mass using the first frame of the currently selected video and may also select crop masks for the processing GUI screen 800. The selected mask may be previewed as shown in FIG. 10 by selection of preview mask 820 of FIG. 8.

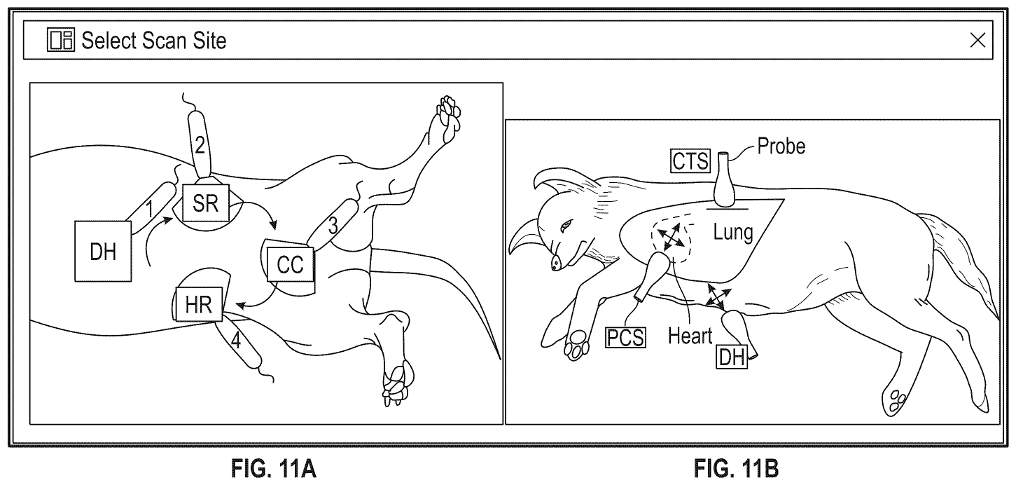

The select site button 824 will open the site selector, as illustrated in FIGS. 11A and 11B (see below) to select a site for the video to be stored under. Augmentation options, illustrated in augmentation buttons 826-838 can be selected along with the percentage of images to which the augmentation will be randomly applied to. For example, grayscale, slip, rotate and respective percentages to be applied are controlled through judicious selection of augmentation buttons 828-838. Progress, % progress fields/buttons 840, 842 allow a user to visually track progress of video processing for a selected ultrasound video.

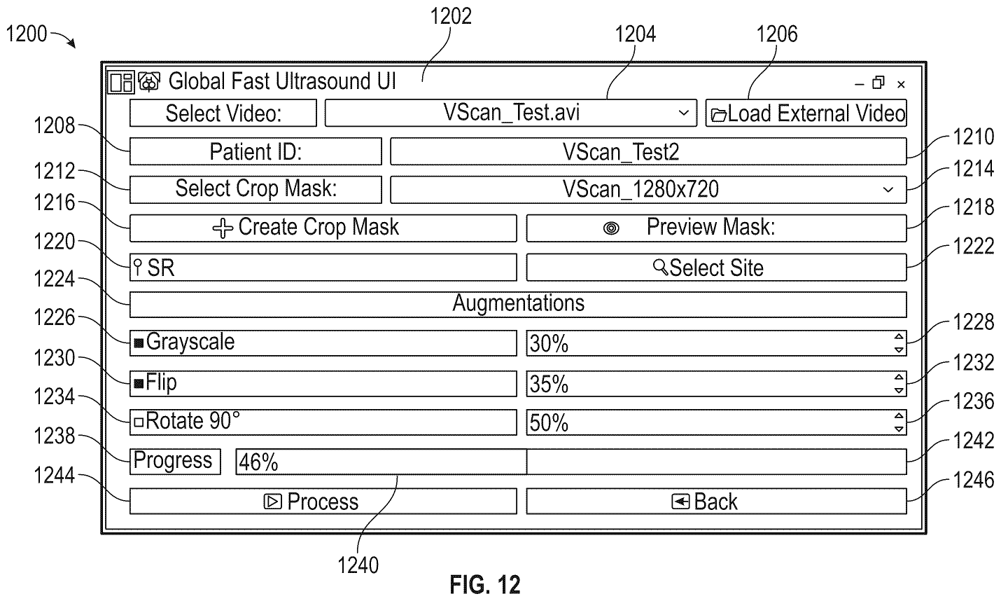

Referring to FIG. 12, a video processing screen 1200 of a Global Fast Ultrasound UI 1202 that is shown to the user of the app via the GUI when the Process Video button 404 is activated (chosen) by the app user; a variety of form fields are filled/selected as shown. In this example, the video processor/controller (shown in FIG. 17) is running in accordance with the selections made by the user, showing the video processing of video VScan_Test.avi 1204. The Patient identification (ID) 1208 is VScan_Test2, the selected crop mask 1212 is VScan_1280×720 selected from dropdown menu 1214. The user may create a crop mask and preview a mask by utilization of buttons/fields 1216 and 1218. The site selected 1220 is SR and can be changed by utilizing select site button 1222. The augmentations 1224 selected as grayscale 1226 at 30% 1228, flip 1230 at 35% 1232; rotate 90 degrees augmentation 1234 at 50% 1236 is not selected. The progress 1238 of the video processing is currently at 46% 1240 in this example. The process button/field 1244 is engaged; the user may go back in the processing by selection of the back button/field 1246.

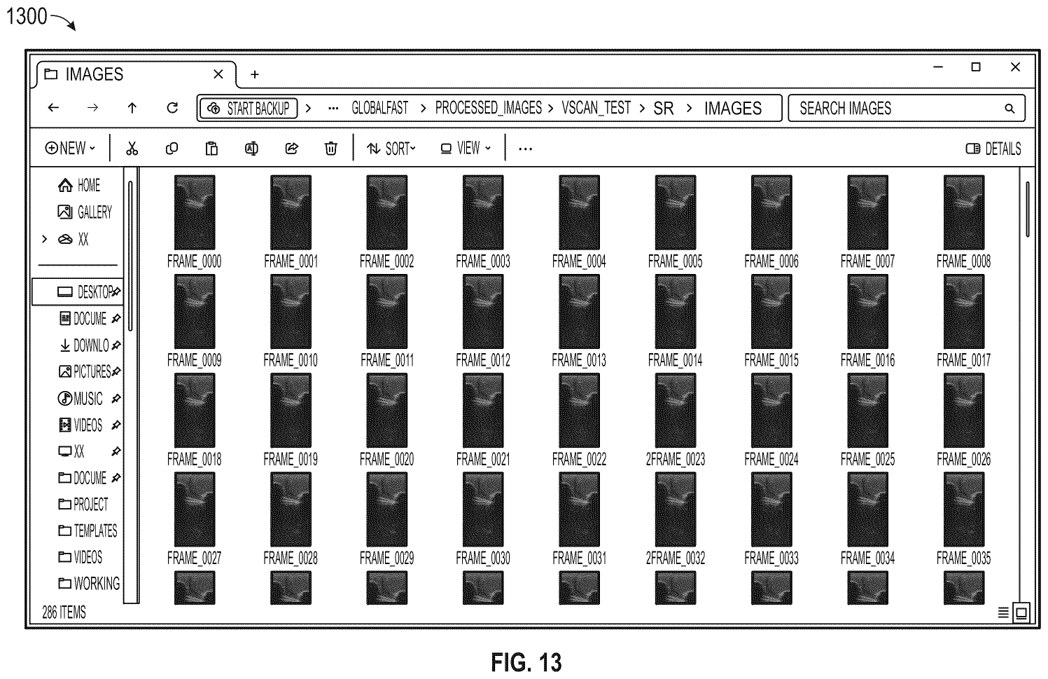

The video processing of scans taken by ultrasound scan are done in accordance with selected sites and augmentation settings. These processed video frames may be saved off in associated database, which may be saved on a mobile, portable device or even in the cloud for later retrieval, as discussed further in connection with FIGS. 17, 18. FIG. 13 shows an example of a folder 1300 containing processed ultrasound video frames, in this example for processed SR ultrasound video frames. In this example, the SR video frames are saved on a directory shown as

### Globalfast/Processed Images/Vscan Test/Sr/Images.

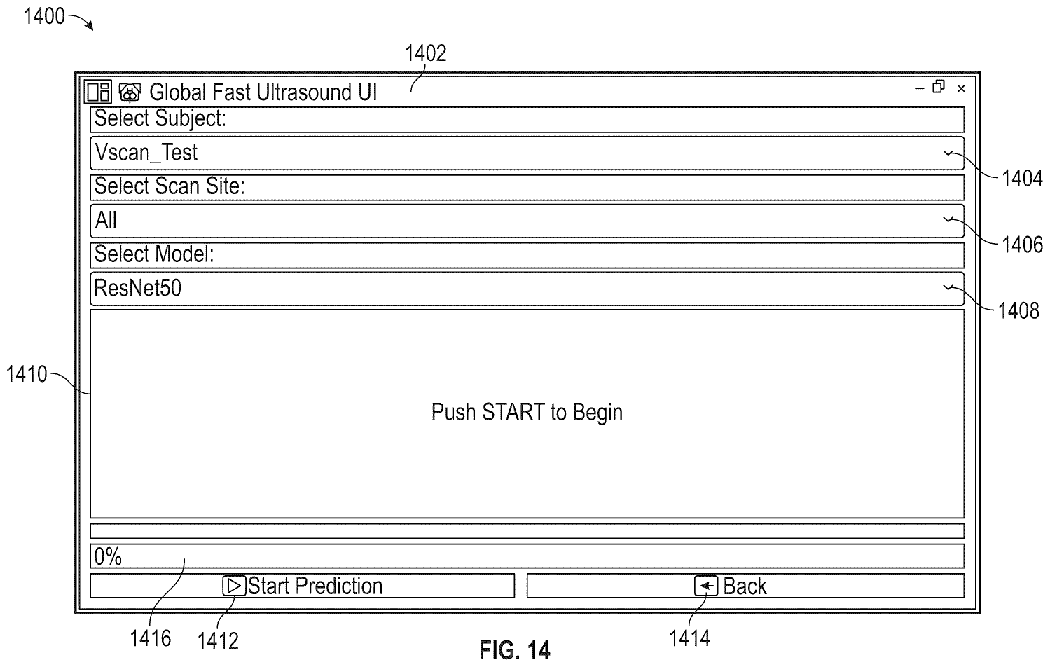

The processed images are used to automatically predict an internal trauma injury of a patient. Selection of Run Predictions button 406 of FIG. 4 presents the predictions screen/page 1400 of a Global Fast Ultrasound UI 1402 of the app to the user, in FIG. 14. The Global Fast Ultrasound UI 1402 predictions page allows the user to Select the Subject 1404, Select the Scan Site 1406, and Select the AI Model 1408. The selected image folder dropdown 1404, 1406 will automatically be populated with the most recently processed data. The site dropdown will be populated with all available sites from within the currently selected image folder. In this example embodiment, the user has selected the Vscan Test file, all scan sites and AI model ResNet50. As instructed by the interface button 1410 of the UI, the user pushes or selected the Start button 1412 to begin the prediction with the progress illustrated in progress bar 1416. The user may go backwards using back button 1414.

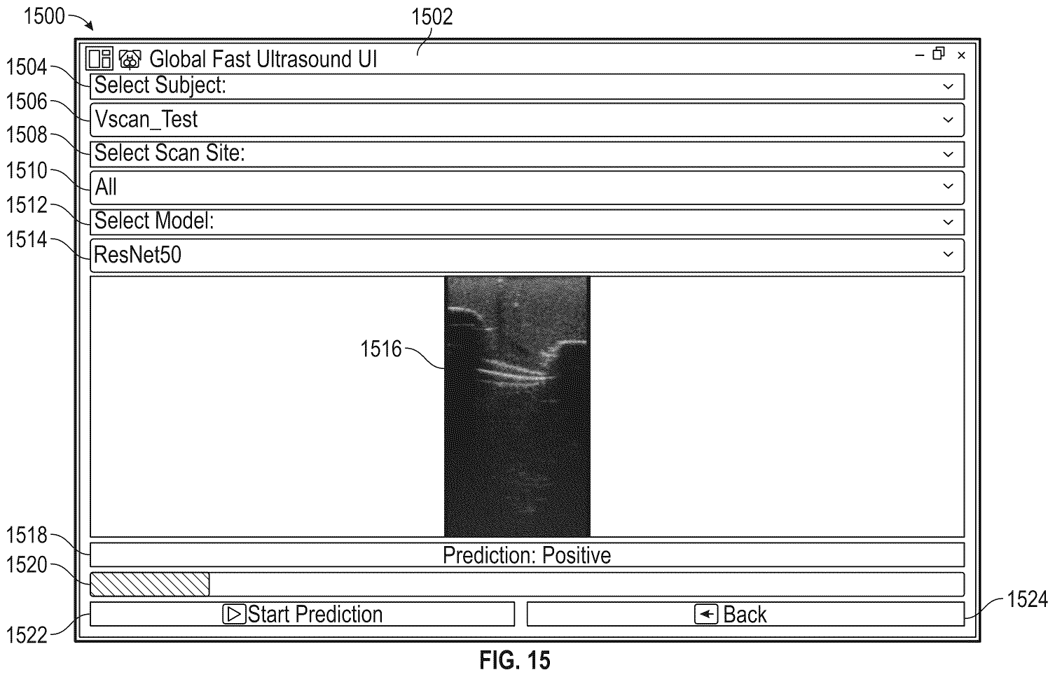

Selection by the user of the start prediction button 1412 causes the user interface of the application to show progress in the prediction of VScan Test file for all selected scan sites, using the ResNet50 AI model. As shown in predictions screen/page 1500 of a Global Fast Ultrasound UI 1502 of FIG. 15, the image 1516 of VScan Test is shown to the user through the (graphical) user interface. Fields 1504-1514 communicate to the user the selected subject 1504, 10506, the selected scan site 1508, 1510, the selected AI model 1512, 1514 (ResNet50 in this example). Start prediction button 1522 has been chosen by the user and the prediction of the current ultrasound image for traumatic injury is positive (field 1518). The user may go backwards by selection of the back button 1524.

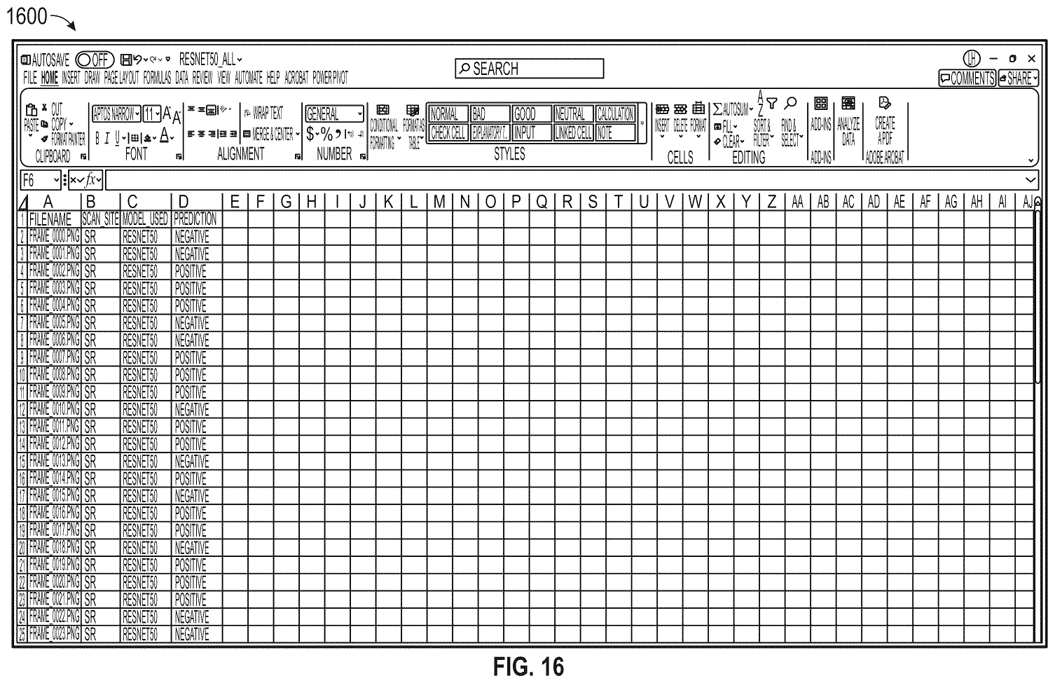

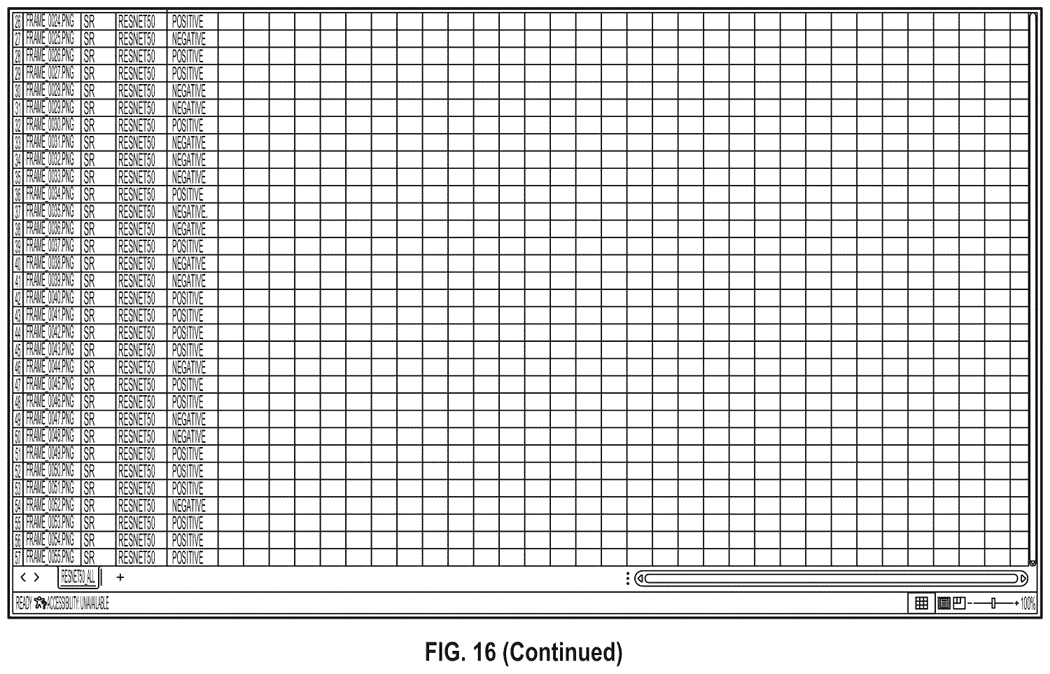

In FIG. 16, the user interface 1600 shows to the user of the application every ten frames during processing to allow time for the user to see the results. The user interface 1600 communicates the prediction of the shown frame in column A, by the scan site SR in column B, the AI model used (RestNet50) in column C, and the prediction for each frame in column D. The progress bar is sown along the bottom of the screen. As shown in FIG. 16, an example csv file is shown to the user. Other forms of communicating the predictions may be used as well.

Referring now to the system and hardware/software for implementing the methods described herein, FIG. 17 illustrates a system diagram of an example environment 1700 for an injury prediction system 1702, in accordance with example embodiments of the disclosure. The system diagram illustrates a series of interactions between one or more devices of the injury prediction system 1702 that is configured to diagnose traumatic internal injuries of an injured subject or patient. The injury prediction system 1702 can be employed by medical and non-medical technicians both on and off the battlefield to rapidly identify hemorrhages or other internal, traumatic injuries of WMDs or other patients to determine on-site treatment options or requests for evacuation.

The injury prediction system 1702 can include a variety of interconnected equipment connected either directly or via a network that provides ultrasound data used to predict internal injuries of a patient. For example, an ultrasound device 1724 having one or more attached transducer probe(s) 1725 can be used to send and receive sounds waves via the piezoelectric effect Specifically, in the transducer probe(s) 1725, there can be one or more quartz piezoelectric crystals that change shape rapidly when an electric current is applied to them. The rapid shape changes, or vibrations, of the crystals produce sound waves that travel outward. Conversely, when sound or pressure waves hit the crystals, they emit electrical currents that can be converted into digital readings. Therefore, the same crystals can be used to send and receive sound waves The transducer probe(s) 1725 also have a sound absorbing substance to eliminate back reflections from the probe itself and an acoustic lens to help focus the emitted sound waves. The electric current applied to crystals to generate sound waves is controlled by a central processing unit (CPU) or processor of the ultrasound device 1724 or other connected computing hardware As such, the CPU sends electrical currents to the transducer probe to emit sound waves. The CPU also receives the electrical pulses from the probes that were created from the returning echoes and generates digital data based on a conversion of these electrical pulse readings. Thus, the CPU can perform all of the calculations involved in processing signals received by a transducer probe 1725. Once the raw signal data are processed, the CPU generates image data which can be stored on the ultrasound device 1724 and displayed on a monitor that is part of the ultrasound device 1724 and/or connected separately. The CPU can also store the image data on a disk or transmit it to the injury prediction system 1702 to be stored in a database 1712 by a data management engine or controller 1704. Also, in one example, the ultrasound device(s) 1724 and injury prediction system 1702 can be part of the same device, which may be a portable device adapted especially useful for forward use in the field.

The signal data received by the probe 1725 are received and processed by the CPU of the ultrasound device 1724 and/or can be transmitted to processing circuitry such as controller 1704 of the injury prediction system 1702 for processing to generate a plurality of sequenced image data stored in image data database 1712. As discussed above, the processing of the ultrasound image data is done per scan site of the patient, Accordingly, image data database 1712 includes CTS or CTS_M data 1714, PCS data 1716, DH data 1718, SR data 1720, CC data 1722, and HR data 1723 as shown.

These images are processed as discussed above by the CPU of the ultrasound device 1724 and/or the processing circuitry, the controller 1704 of the injury prediction system 1702, to generate a time-sequence video of blood flow and tissue movement especially near their interface as image data. The time-sequence, either as a whole, and/or on a frame-by-frame basis, is then analyzed by an image processing engine 1708 to diagnose the extent of an injury. For example, in one implementation in color mode, the mean velocity within a pixel-divided region is determined based on a frame-by-frame comparison. The mean-velocity is then converted into a color, such as blue, when blood is flowing toward the transducer probe and red when blood is flowing away from the transducer probe. These colors are then shown on the display of the ultrasound device 1724 and/or the injury prediction system 1702 and/or remote device(s) 1726/1730 to illustrate the blood flow and tissue movements and to highlight a potential area of injury.

In one example, once the transducer probe 1725 beam has vibrated any blood and nearby tissue of the patient, the signals are received by the injury prediction system 1702 either via direct connection to the ultrasound device 1724 or wirelessly through a network. The injury prediction system 1702 can also receive the image data generated by the ultrasound device 1724 or can in some implementations receive signal data of the transducer probe 1725 and generate the image data internally via an image generation engine or circuit 1706 of injury prediction system 1702. In accordance with some embodiments, for example in a segmented system implementation, ultrasound image generation at a remote injury prediction system 1702 allows the ultrasound device 1724 to require less computational equipment thereby reducing the size and footprint of the ultrasound device. This can help soldiers or trauma technicians by providing a more compact ultrasound device that can be easily deployed in the field. Once the ultrasound image data are either received and/or generated by the injury prediction system 1702, the data are stored in a image database 1712 by the data management engine or controller 1704. The data management engine or controller 1704 is responsible for handling incoming, outgoing and internal data controlled by the injury prediction system 1702. Accordingly, the data management engine or controller 1704 passes the signal data received by the ultrasound device 1724 to the image generation engine or circuitry 1706 for processing, described above, and provides the generated image data from image data database 1712 to the image processing engine/circuit 1708 for processing while also managing the storing and retrieval of processed data in image data database 1712.

The image processing engine/circuit 1708 processes the image data received by the ultrasound 1724 and/or generated by the image generation engine 1706 to diagnose an injury of a patient. To do this, the image processing engine/circuit 1708 segments the image data on a frame-by-frame basis to identify areas of color or specific blood flow to determine whether there are pools of blood indicating internal trauma conditions such as hemorrhaging. For example, blood between two organs, such as the kidney and liver, would result in both the liver and kidney surfaces being highlighted in images of corresponding scan sites of the patient. The amount of flow detected by ultrasound can be analyzed by the injury prediction engine or circuit 1711 by scan site and in accordance with selected augmentation settings using one or more trained AI models to automatically generate a predicted internal trauma injury results at a selected scan site of the patient. as described above. The predicted internal trauma injury result(s) can be provided, such as to a user of the system via a user interface of an app used by the user, by notification engine 1710.

Report data of the prediction made by injury prediction engine 1711 and communicated by notification engine 1710 may be stored in a database or server for later retrieval as needed. Accordingly, notification engine 1710 of the injury prediction system 1702 generates and stores report data and presents this to the technician via a display screen of the injury prediction system 1702, on a remote computing device 126 and/or on a screen of the ultrasound device 1724. The report data can provide time-linked images as a video or specific time-sliced images specific to the analyzed area of the patient and including color halos generated by Doppler, a probability prediction of how likely it is that the patient is suffering from internal abnormalities, such as bleeding via a hemorrhage, and diagnosis and alert information regarding the lethality of the injury and potential actions that the technician can take based on the diagnosis. These actions can include on-site surgical treatments and instructions specific to the diagnosis or instructions that the patient should be moved immediately and an indication of the closest medical facility for treatment. In one example, image data of the patient, such as a soldier, can be pre-stored in the image database 1712 or the injury prediction system 1702 can retrieve and store image data from one or more devices worn by the patient or remote devices 126-130. Alternatively, or in addition to a computer-generated report, the technician themselves can review the image data and report data to provide an assessment of the injury of the patient. However, this is not always easy, especially in a battlefield environment, so the computer-generated report including prediction of trauma injury by scan site of a patient can save time while also providing life-saving treatment options thereby greatly increasing survivability of the injured patient.

One or more computing devices 1726, one or more servers 1728 and one or more mobile devices 1730 can also be connected to the injury prediction system 1702 either directly or via a network. The computing devices 1726 and/or mobile devices 1730 can be used to remotely receive and review image data, analysis data and report data transmitted by the data management engine or controller 1704. Accordingly, remote technicians can review data that is being provided by the injury prediction system 1702 in real-time to allow remote medical experts to provide additional support for the injured patient. For example, a soldier on the battlefield with no medical expertise could receive instruction from remote medical experts on treating the injured soldier. The one or more servers 1728 can also help provide for additional processing of the signal data and image data and can further act to push software and firmware updates as well as updated image data to the injury prediction system 1702.

As used herein data management engine or controller 1704 can be a processor or processors that include one or more general-purpose or application-specific microprocessors that executes instructions to perform control, computation, input/output, etc. functions for injury protection system 1702. Such a processor may include a single integrated circuit, such as a micro-processing device, multiple cores, multiple integrated circuit devices and/or circuit boards working in cooperation, etc., to accomplish the functions of a processor/controller. In addition, the controller may execute computer programs or modules, such as operating system, software modules, etc., stored within a memory. For example, software modules may include an AI application, an ML application, an ANN application, a CNN application, etc. Generally, the controller/processor 1704 includes one or more instruction decoders; in many embodiments, it may include an instruction decoder configured to control processing circuitry to perform the scatter accumulate instruction.

Generally, storage element or memory stores instructions for execution by controller/processor and data. Memory may include a variety of non-transitory computer-readable medium that may be accessed by the controller/processor. In various embodiments, memory may include volatile and nonvolatile medium, non-removable medium and/or removable medium. For example, memory may include any combination of random access memory (RAM), dynamic RAM (DRAM), static RAM (SRAM), read only memory (ROM), flash memory, cache memory, and/or any other type of non-transitory computer-readable medium.

Memory contains various components for retrieving, presenting, modifying, and storing data. For example, memory stores software modules that provide functionality when executed by controller/processor 1704. The software modules include operating system that provides operating system functionality for a computer, for example. Software modules may provide various functionality, such as image classification using convolutional neural networks, etc. Data may include data associated with operating system, software modules, etc.

The functions and features described herein may also be executed by various distributed components of a system. For example, one or more processors may execute these system functions, with processors distributed across multiple components communicating in a network. The distributed components may include one or more client and server machines, which may share processing, as shown in FIG. 18, in addition to various human interface and communication devices (e.g., display monitors, smart phones, tablets, personal digital assistants (PDAs)). The network may be a private network, such as a LAN or WAN, or may be a public network, such as the Internet. Input to the system may be received via direct user input via an application running on the system and received remotely either in real-time or as a batch process. Additionally, some implementations may be performed on modules or hardware not identical to those described. Accordingly, other implementations are within the scope that may be claimed.

Referring now to FIG. 18, a block diagram 1800 shows an example of cloud computing, with various devices interconnected to each other via a network and cloud infrastructures. Injury prediction system 1802, similar to 1702 of FIG. 17, can be in communication with such a cloud-based network 1800. FIG. 18 shows user devices such as a tablet 1812 and a cellular phone 1814 connected to the mobile network service 1820 through a wireless access point 1854, such as a Wi-Fi network. Further, FIG. 18 shows the injury diagnosis system 1802 connected to the mobile network service 1820 through a wireless channel using a base station 1856, such as an Edge, 3G, 4G, 5G, LTE® or similar network, for example. Various other permutations and types of communications between types of devices and mobile network service 1820 are also possible, as would be understood to one of ordinary skill in the art. Various types of devices, such as the cellular phone 1814, a tablet computer, or a desktop computer 1801, can also access the network 1840 and the cloud 1830 through a fixed/wired connection, such as through a USB connection to a desktop or laptop computer or workstation that is connected to the network 1840 via a network controller, such as a network interface card for interfacing with a network.

Signals from the wireless interfaces (e.g., the base station 1856, the wireless access point 1854, and the satellite connection 1852) are transmitted to and from the mobile network service 1820. Requests from mobile users and their corresponding information as well as information being sent to users is transmitted to central processors 1822 that are connected to servers 1824 providing mobile network services, for example. Further, mobile network operators can provide services to the various types of devices. For example, these services can include authentication, authorization, and accounting based on home agent and subscribers' data stored in databases 1826, for example. The subscribers' requests can be delivered to the cloud 1830 through network 1840.

As can be appreciated, the network 1840 can be a public network, such as the Internet, or a private network such as an LAN or WAN network, or any combination thereof and can also include PSTN or ISDN sub-networks. The network 1840 can also be a wired network, such as an Ethernet network, or can be a wireless network such as a cellular network including EDGE, 3G and 4G wireless cellular systems. The wireless network can also be Wi-Fi, Bluetooth®, or any other wireless form of a communication that is known.

The various types of devices can each connect via the network 1840 to the cloud 1830, receive inputs from the cloud 1830 and transmit data to the cloud 1830. These cloud services are provided using concepts of utility computing, virtualization, and service-oriented architecture. Data from the cloud 1830 can be accessed by the injury diagnosis system 1802 based on user interaction and pushed to user devices such as 1810, 1812, and 1814.

The cloud 1830 can be accessed via a user interface such as a secure gateway 1832. The secure gateway 1832 can, for example, provide security policy enforcement points placed between cloud service consumers and cloud service providers to interject enterprise security policies as the cloud-based resources are accessed. Further, the secure gateway 1832 can consolidate multiple types of security policy enforcement, including, for example, authentication, single sign-on, authorization, security token mapping, encryption, tokenization, logging, alerting, and API control. The cloud 1830 can provide to users computational resources using a system of virtualization, in which processing and memory requirements can be dynamically allocated and dispersed among a combination of processors and memories such that the provisioning of computational resources is hidden from the users and making the provisioning appear seamless as though performed on a single machine. Thus, a virtual machine is created that dynamically allocates resources and is therefore more efficient at utilizing available resources. A system of virtualization using virtual machines creates an appearance of using a single seamless computer even though multiple computational resources and memories can be utilized according increases or decreases in demand. The virtual machines can be achieved using a provisioning tool 1829 that prepares and equips the cloud-based resources such as a processing center 1834 and data storage 1838 to provide services to the users of the cloud 1830. The processing center 1834 can be a computer cluster, a data center, a main frame computer, or a server farm. The processing center 1834 and data storage 1838 can also be collocated.

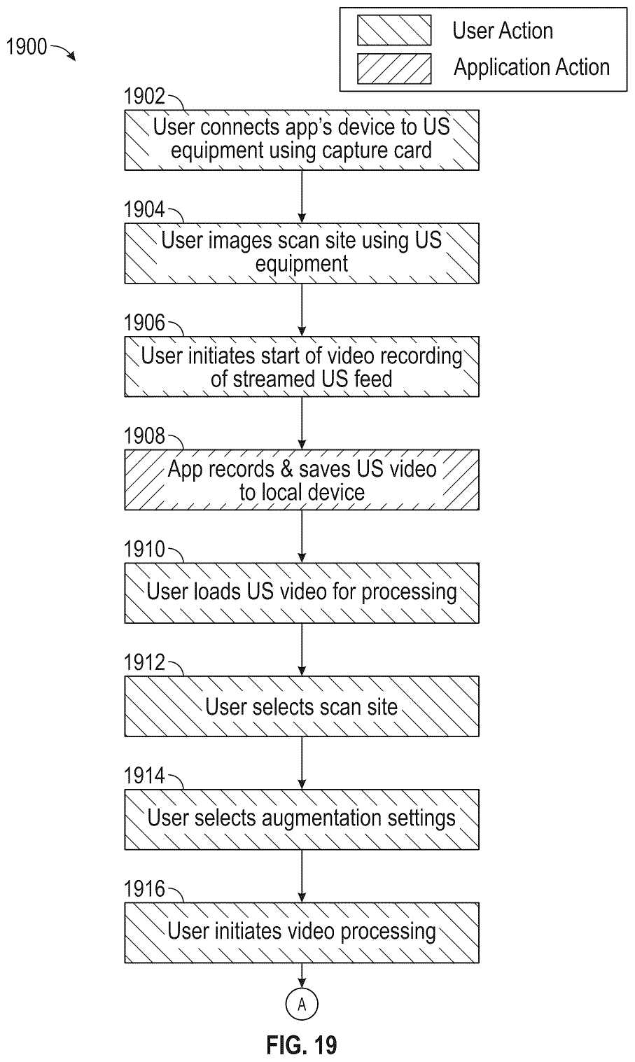

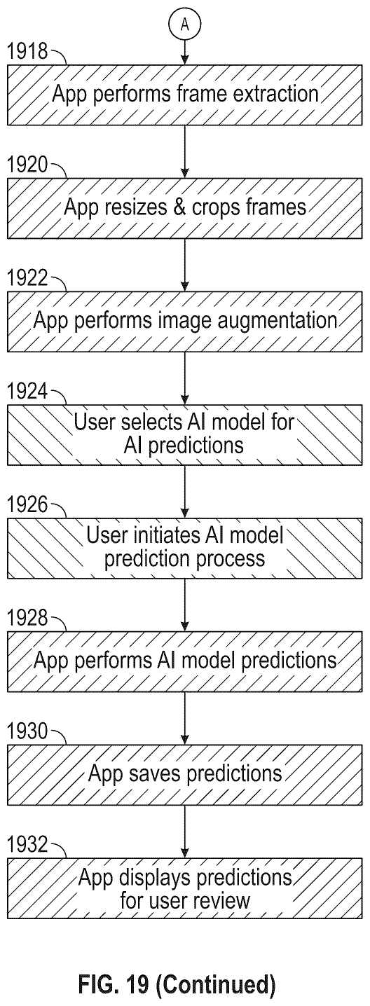

Referring now to FIG. 19, a flow 1900 for predicting internal trauma of a patient using ultrasound and trained AI models is shown. As indicated by the hashing in the drawing, some of these actions are by a user of a prediction system, such as via an interface of an app (lication) that runs on the system, as previously described at length. Other actions are the purview of the application itself and are performed automatically once guidelines for analysis (processing) and prediction are supplied. At block 1902, a user connects the application's device to ultrasound equipment (US) using a capture card or the like. As previously described, the US equipment may be a portable, handheld device having US capabilities. At block 1904, the user performs an ultrasound image scan at one or more scan sites of a patient using the US enabled equipment. At block 1906, the user initiated via the app the start of the video recording of the streamed US feed provided by the US equipment during an US scan of one or more scan sites of the patient. At block 1908, the app records and saves the US video to a local device; additionally, this information may be remotely stored, such as transmitted to storage of a cloud computing network for later retrieval and use. At block 1910, the user loads the US video(s) for processing. At block 1912, the user selects the scan site to be processes and the augmentation settings for processing at block 1914. The user initiates video processing at block 1916. Responsive to the user initiating video processing, the app performs frame extraction at block 1918, resizes and crops extracted frames at block 1920, and performs image augmentation at block 1922.

At block 1924, the user selects the AI model(s) to be used to making AI predictions of injuries of selected US images and initiates the AI model prediction process at block 1926. The app performs the AI model predictions in accordance with the selections made by the user at block 1928 and saves the predictions at block 1930 to storage, on the US device and/or to remote storage of the system. Finally, at block 1932 the app displays the prediction results to the user for user review.

Embodiments of the invention have been described to explain the nature of the invention. Those skilled in the art may make changes in the details, materials, steps and arrangement of the described embodiments within the principle and scope of the invention, as expressed in the appended claims.

While implementations of the disclosure are susceptible to embodiment in many different forms, there is shown in the drawings and will herein be described in detail specific embodiments, with the understanding that the present disclosure is to be considered as an example of the principles of the disclosure and not intended to limit the disclosure to the specific embodiments shown and described. In the description above, like reference numerals may be used to describe the same, similar or corresponding parts in the several views of the drawings.

In this document, relational terms such as first and second, top and bottom, and the like may be used solely to distinguish one entity or action from another entity or action without necessarily requiring or implying any actual such relationship or order between such entities or actions. The terms “comprises,” “comprising,” “includes,” “including,” “has,” “having,” or any other variations thereof, are intended to cover a non-exclusive inclusion, such that a process, method, article, or apparatus that comprises a list of elements does not include only those elements but may include other elements not expressly listed or inherent to such process, method, article, or apparatus. An element preceded by “comprises . . . a” does not, without more constraints, preclude the existence of additional identical elements in the process, method, article, or apparatus that comprises the element.

Reference throughout this document to “one embodiment,” “certain embodiments,” “an embodiment,” “implementation(s),” “aspect(s),” or similar terms means that a particular feature, structure, or characteristic described in connection with the embodiment is included in at least one embodiment of the present disclosure. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means “any of the following: A; B; C; A and B; A and C; B and C; A, B and C.” An exception to this definition will occur only when a combination of elements, functions, steps or acts are in some way inherently mutually exclusive. Also, grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth. References to items in the singular should be understood to include items in the plural, and vice versa, unless explicitly stated otherwise or clear from the text.

Recitation of ranges of values herein are not intended to be limiting, referring instead individually to any and all values falling within the range, unless otherwise indicated, and each separate value within such a range is incorporated into the specification as if it were individually recited herein. The words “about,” “approximately,” or the like, when accompanying a numerical value, are to be construed as indicating a deviation as would be appreciated by one of ordinary skill in the art to operate satisfactorily for an intended purpose. Ranges of values and/or numeric values are provided herein as examples only, and do not constitute a limitation on the scope of the described embodiments. The use of any and all examples, or exemplary language (“e.g.,” “such as,” “for example,” or the like) provided herein, is intended merely to better illuminate the embodiments and does not pose a limitation on the scope of the embodiments. No language in the specification should be construed as indicating any unclaimed element as essential to the practice of the embodiments.

For simplicity and clarity of illustration, reference numerals may be repeated among the figures to indicate corresponding or analogous elements. Numerous details are set forth to provide an understanding of the embodiments described herein. The embodiments may be practiced without these details. In other instances, well-known methods, procedures, and components have not been described in detail to avoid obscuring the embodiments described. The description is not to be considered as limited to the scope of the embodiments described herein.

In the following description, it is understood that terms such as “first,” “second,” “top,” “bottom,” “up,” “down,” “above,” “below,” and the like, are words of convenience and are not to be construed as limiting terms. Also, the terms apparatus, device, system, etc. may be used interchangeably in this text.

The many features and advantages of the disclosure are apparent from the detailed specification, and, thus, it is intended by the appended claims to cover all such features and advantages of the disclosure which fall within the scope of the disclosure. Further, since numerous modifications and variations will readily occur to those skilled in the art, it is not desired to limit the disclosure to the exact construction and operation illustrated and described, and, accordingly, all suitable modifications and equivalents may be resorted to that fall within the scope of the disclosure.

What is claimed is:

1. A method for automatically predicting an internal trauma of a patient, comprising:
processing point of care ultrasound (POCUS) images of the patient, each POCUS image associated with a scan site of the patient, in accordance with one or more selected scan sites of the patient and augmentation settings to generate processed POCUS images of the patient; and
interpreting the processed POCUS images of the patient associated with the one or more selected scan sites and augmentation settings using one or more trained AI models to automatically generate a predicted internal trauma injury result at a selected scan site of the patient.

2. The method of claim 1, further including recording an ultrasound video of the POCUS images captured during an ultrasound scan of the patient, where the POCUS images are from ultrasound clips of the ultrasound video.

3. The method of claim 2, where the processing of POCUS images of the patient is performed during one or more of in real-time with the recording the ultrasound video of the POCUS images captured during an ultrasound scan of the patient and
after the recording the ultrasound video of the POCUS images where the recorded ultrasound video of the POCUS images are loaded and processed by a processing circuit of a system for predicting an internal trauma injury of the patient.

4. The method of claim 1, where the processing POCUS images of the patient further includes pre-processing for each POCUS image associated with a scan site of the patient including one or more of:
capturing from a POCUS ultrasound video of the patient, one or more POCUS ultrasound video clips with each POCUS ultrasound video clip associated with a scan site of the one or more scan sites of the subject;
extracting from each POCUS ultrasound video clip POCUS frames;
cropping and resizing the extracted POCUS frames; and
augmenting the POCUS frames to generate the plurality of processed POCUS images of the patient in accordance with augmentation settings.

5. The method of claim 4, where the processing POCUS images of the patient is performed by an application controlled by a processor of a system for diagnosing a patient and where a user of the system loads the POCUS ultrasound video of the patient, selects the scan site of each POCUS ultrasound video clip, selects the augmentation settings, and initiates the processing POCUS images via a user interface of the application.

6. The method of claim 4, further including processing two-dimensional images to create reconstructed M-mode images and cropping and resizing the reconstructed M-mode images.

7. The method of claim 1, further including generating mask overlays of the scan site datasets to refine generated predicted internal trauma injury results.

8. The method of claim 1, where the one or more trained AI models are trained by deep learning classification neural networks using a plurality of captured ultrasound images and further comprising training the one or more trained AI models including:
splitting a plurality of ultrasound images of a plurality of subjects into scan site datasets with each scan site dataset defined by the scan site of the plurality of ultrasound images, each ultrasound image associated with a scan site of the plurality of scan sites of a subject of the plurality of subjects;
training a trained AI model of the one or more trained AI models on the scan site datasets to generate predicted internal trauma injury results on the scan site datasets.

9. The method of claim 8, further including prior to splitting the plurality of ultrasound images of the plurality of subjects into scan site datasets:
recording for each subject of the plurality of subjects an ultrasound scan of the subject at one or more scan sites of the subject;
capturing from the ultrasound scan, one or more ultrasound video clips with each ultrasound video clip associated with a scan site of the one or more scan sites of the subject;
extracting from each ultrasound video clip frames;
sorting each extracted frame by positive or negative for an internal trauma injury at the scan site of the extracted frame; and
processing each extracting frame to generate the plurality of ultrasound images of the plurality of subjects.

10. The method of claim 9, further including one or more of exporting the one or more ultrasound video clips for processing, cropping one or more extracted and sorted ultrasound video clips in accordance with a crop mask overlay, and augmenting the plurality of ultrasound images of the plurality of subjects prior to training the trained AI model.

11. The method of claim 9, further including processing two-dimensional images to create reconstructed M-mode images and cropping and resizing the reconstructed M-mode images.

12. A system for automatically predicting an internal trauma of a patient, comprising:
a controller;
a processing circuit controlled by the controller and configured to process a plurality of point of care ultrasound (POCUS) images of the patient in accordance with one or more selected scan sites of the patient and augmentation settings to generate processed POCUS images of the patient, each POCUS image associated with a scan site of the patient; and
a prediction circuit controlled by the controller and configured to interpret the processed POCUS images of the patient associated with the one or more selected scan sites and augmentation settings using one or more trained AI models to automatically generate a predicted internal trauma injury result at a selected scan site of the patient, where the controller controls the prediction circuit to communicate the predicted internal trauma injury result through a user interface of the system controlled by the controller.

13. The system of claim 12, the system further comprising:
an ultrasonic probe controlled by the controller and configured to record the plurality of POCUS images of the patient captured during an ultrasound scan of the patient, the processing circuit configured to receive the plurality of POCUS images from the ultrasonic probe.

14. The system of claim 13, where the processing circuit processes POCUS images of the patient during one or more of in real-time with the ultrasound probe recording the ultrasound video of the POCUS images captured during the ultrasound scan of the patient by the ultrasonic probe and
after the ultrasonic probe records the ultrasound video of the POCUS images in which the processor controls loading of the recorded ultrasound video of the POCUS images from storage to the processing circuit of the system for predicting an internal trauma injury of the patient.

15. The system of claim 12, where the processing circuit is configured for each POCUS image associated with a scan site of the patient to:
capture from a POCUS ultrasound video of the patient, one or more POCUS ultrasound video clips with each POCUS ultrasound video clip associated with a scan site of the one or more scan sites of the subject;
extract from each POCUS ultrasound video clip POCUS frames;
crop and resize the extracted POCUS frames; and
augment the POCUS frames to generate the plurality of processed POCUS images of the patient in accordance with augmentation settings.

16. The system of claim 15, where a user of the system loads the POCUS ultrasound video of the patient, selects the scan site of each POCUS ultrasound video clip, selects the augmentation settings, and initiates the processing POCUS images via a user interface of an application running on the system and in operable communication with and controlled by the controller.

17. The system of claim 16, where the system is a handheld ultrasound system.

18. The system of claim 15, where the processing circuit is configured for each POCUS image associated with a scan site of the patient to process in accordance with a crop mask overlay.

19. The system of claim 18, where a user of the system selects the crop mask overlay from a plurality of crop mask overlays via a user interface of the system in operable communication with the controller.

20. The system of claim 15, where the processing circuit is configured to process two-dimensional images to create reconstructed M-mode images and crop and resize the reconstructed M-mode images.
